(19) United States
(12) Patent Application Publication
(10) Pub. No.: US 2013/0253019 A1
(43) Pub. Date: Sep. 26, 2013

(54) LYSOPHOSPHATIDIC ACID RECEPTOR ANTAGONISTS FOR THE TREATMENT OF CONDITIONS OR DISEASES OF THE EYE

(71) Applicants: John Howard Hutchinson, San Diego, CA (US); Thomas Jon Seiders, San Diego, CA (US); James Stephen Swaney, Carlsbad, CA (US)

(72) Inventors: John Howard Hutchinson, San Diego, CA (US); Thomas Jon Seiders, San Diego, CA (US); James Stephen Swaney, Carlsbad, CA (US)

(21) Appl. No.: 13/704,276

(22) PCT Filed: Jun. 13, 2011
(86) PCT No.: PCT/US11/40230

§ 371 Date: Apr. 29, 2013

Related U.S. Application Data
(60) Provisional application No. 61/355,102, filed on Jun. 15, 2010.

(51) Int. Cl. A61K 31/42; A61K 31/4245

(52) U.S. Cl. CPC A61K 31/42; A61K 31/4245
USPC 514/364; 514/380

(57) ABSTRACT

Described herein is the use of LPA1 antagonists in the treatment or prevention of diseases or conditions of the eye of a mammal. Also described are pharmaceutical compositions that include at least one LPA1 antagonist.

### RELATED APPLICATIONS

This application claims the benefit of U.S. provisional patent application No. 61/355,102 entitled “LYSOPHOSPHATIDIC ACID RECEPTOR ANTAGONISTS FOR THE TREATMENT OF CONDITIONS OR DISEASES OF THE EYE” filed on Jun. 15, 2010, which is incorporated by reference in its entirety.

### FIELD OF THE INVENTION

Described herein are methods of treatment and ophthalmic formulations comprising Lysophosphatidic acid (LPA) receptor antagonists, wherein the LPA receptor antagonists are used to treat or prevent diseases, disorders or conditions mediated by one or more of the LPA receptors.

### BACKGROUND OF THE INVENTION

Lysophospholipids are membrane-derived bioactive lipid mediators. Lysophospholipids affect fundamental cellular functions that include proliferation, differentiation, survival, migration, adhesion, invasion, and morphogensis. These functions influence many biological processes that include, but are not limited to, wound healing, fibrosis, angiogenesis, immunity, neurogenesis, and carcinogenesis.

Lysophophatidic acid (LPA) is a lysophospholipid that has been shown to act through sets of specific G-protein-coupled receptors (GPCRs) in an autocrine and paracrine fashion. LPA binding to its cognate G-protein coupled receptors (LPA₁, LPA₂, LPA₃, LPA₄, LPA₅, LPA₆) activates intracellular signaling pathways and produces a variety of biological responses. Antagonists of the LPA receptors, administered to any ocular site of a mammal using a suitable formulation or device, are used to prevent, ameliorate, or treat LPA-dependent or LPA-mediated diseases or conditions.

### SUMMARY OF THE INVENTION

In one aspect, presented herein are compounds that inhibit the physiological activity of lysophosphatidic acid (LPA), and are useful as agents for the treatment or prevention of ocular diseases or conditions or diseases or conditions in which ocular tissues are affected. In some embodiments, such diseases or conditions will benefit from inhibition of the physiological activity of LPA, such as diseases in which an LPA receptor participates, is involved in the etiology or pathology of the disease, or is otherwise associated with at least one symptom of the disease. Such diseases or conditions include, but are not limited to dry age related macular degeneration, geographic atrophy, wet age related macular degeneration (wet AMD), wet AMD with neovascularization, wet AMD with foveal thickening, diabetic retinopathy, diabetic retinopathy with retinal edema, diabetic retinopathy with neovascularization, retinitis pigmentosa and other retinal degenerative diseases, proliferative vitreoretinopathy (PVR), prevention and treatment of macular thickening related to photocoagulation, retinopathy of prematurity (ROP), retinal detachment, retinal detachment following penetrating injury)post-surgical macular edema, posterior uveitis, macular edema associated with inherited retinal disease, chronic retinal macular edema, Usher syndrome, Bardet-Biedl syndrome (BBS), branch retinal vein occlusion (BRVO), central retinal vein occlusion (CRVO), ocular hypertension or primary open-angle glaucoma, episcleral fibrosis leading to trabeculectormy (bleb) failure after glaucoma filtration surgery, dry eyes, Sjogren syndrome, inflammation following ocular surgery, keratoconjuctivitis, pterygia, non-specific orbital inflammation, cataracts, cataracts (post-surgical scarring), corneal scarring, scarring associated with cular ciatricial pemphigoid, glaucoma filtration surgery, thyroid eye disease, anterior uveitis, or fibrosis associated with keratoprosthesis procedure.

In some embodiments, provided is a method of treating an ocular disease or condition in a mammal, comprising administering to the mammal in need thereof a therapeutically-effective amount of an LPA1 receptor antagonist.

In some embodiments, the ocular disease or condition is LPA-dependent or LPA-mediated.

In some embodiments, the ocular disease or condition is LPA1-dependent or LPA1-mediated.

In some embodiments, the ocular disease or condition is an ocular disease or condition affecting the posterior segment of the eye, the anterior segment of the eye, or both the posterior segment of the eye and the anterior segment of the eye.

In some embodiments, the ocular disease or condition is an ocular disease or condition affecting the posterior segment of the eye.

In some embodiments, the ocular disease or condition is dry age related macular degeneration, geographic atrophy, wet age related macular degeneration, wet age related macular degeneration with neovascularization, wet age related macular degeneration with foveal thickening, diabetic retinopathy, diabetic retinopathy with retinal edema, diabetic retinopathy with neovascularization, retinitis pigmentosa, retinal degenerative diseases, proliferative vitreoretinopathy (PVR), prevention and treatment of macular thickening related to photocoagulation, retinopathy of prematurity (ROP), retinal detachment, post-surgical macular edema, posterior uveitis, macular edema associated with inherited retinal disease, chronic retinal macular edema, Usher syndrome, Bardet-Biedl syndrome (BBS), Branch retinal vein occlusion (BRVO), or Central retinal vein occlusion (CRVO).

In some embodiments, the ocular disease or condition is an ocular disease or condition affecting the anterior segment of the eye.

In some embodiments, the ocular disease or condition is ocular hypertension, primary open-angle glaucoma, episcleral fibrosis leading to trabeculectormy (bleb) failure after glaucoma filtration surgery, dry eyes, Sjogren syndrome, inflammation following ocular surgery, keratoconjuctivitis, pterygia, non-specific orbital inflammation, cataracts, post-surgical corneal scarring, corneal scarring, scarring associated with ocular cicatricial pemphigoid, glaucoma filtration surgery, thyroid eye disease, anterior uveitis, or fibrosis associated with keratoprosthesis procedure.

In some embodiments, the LPA1 receptor antagonist has a structure of Formula (I), Formula (II), Formula (III), Formula (IV), Formula (V), Formula (VI) or Formula (VII); or a pharmaceutically acceptable salt, prodrug, active metabolite, or a pharmaceutically acceptable solvate thereof. In some embodiments, the LPA1 receptor antagonist has a structure of Formula (I), or a pharmaceutically acceptable salt thereof. In some embodiments, the LPA1 receptor antagonist has a structure of Formula (II), or a pharmaceutically acceptable salt thereof. In some embodiments, the LPA1 receptor antagonist has a structure of Formula (III), or a pharmaceutically acceptable salt thereof. In some embodiments, the LPA1 receptor antagonist has a structure of Formula (IV), or a pharmaceutically acceptable salt thereof. In some embodiments, the LPA1 receptor antagonist has a structure of Formula (V), or a pharmaceutically acceptable salt thereof. In some embodiments, the LPA1 receptor antagonist has a structure of Formula (VI) or a pharmaceutically acceptable salt thereof. In some embodiments, the LPA1 receptor antagonist has a structure of Formula (VII) or a pharmaceutically acceptable salt thereof.

In some embodiments, the LPA1 receptor antagonist is selected from LPA1 receptor antagonists disclosed in: U.S. Provisional Application No. 61/122,568; U.S. Provisional Application No. 61/183,785; U.S. patent application Ser. No. 12/638,702; U.S. Provisional Application No. 61/121,862; U.S. Provisional Application No. 61/231,282; U.S. Provisional Application No. 61/247,681; U.S. Provisional Application No. 61/247,2877; International patent application no. PCT/US2010/44284; International patent application no. PCT/US2010/51199; International patent application no. PCT/US2010/51150; U.S. patent application Ser. No. 12/896,080; International patent application no. PCT/US2010/50786; International patent application no. PCT/US2010/50787; U.S. patent application Ser. No. 12/893,902; International patent application no. PCT/US09/68106; International patent application no. PCT/US09/68105; International patent application no. PCT/US09/67527; International patent application no. PCT/US10/37309; International patent application no. PCT/US10/37316; or U.S. patent application Ser. No. 12/793,440; each of which is herein incorporated by reference.

In some embodiments, the LPA1 receptor antagonist is (R)-2-(4′-(3-methyl-4-((1-phenylethoxy)carbonylamino)isoxazol-5-yl)biphenyl-4-yl)acetic acid (Compound A); (R)-1-(4′-(3-methyl-4-((1-phenylethoxy)carbonylamino)isoxazol-5-yl)biphenyl-4-yl)cyclopropanecarboxylic acid (Compound B); (R)-2-(4′-(4-((1-(2-chlorophenyl)ethoxy)carbonylamino)-3-methylisoxazol-5-yl)biphenyl-4-yl)acetic acid (Compound C); {5-[4′-(1-Methanesulfonylaminocarbonyl-cyclopropyl)-biphenyl-4-yl]-3-methyl-isoxazol-4-yl}-carbamic acid (R)-1-phenyl-ethyl ester (Compound D); 1-(4′-{4-[(R)-1-(2-Chloro-phenyl)-ethoxycarbonylamino]-3-methyl-isoxazol-5-yl}-biphenyl-4-yl)-cyclopropanecarboxylic acid (Compound E); 1-{4′4-[4((R)-1-Phenyl-ethoxycarbonylamino)-isoxazol-5-yl]-biphenyl-4-yl}-cyclopropanecarboxylic acid (Compound F); (3-Methyl-5-{4′-[1-(5-oxo-2,5-dihydro-[1,2,4]oxadiazol-3-yl)-cyclopropyl]-biphenyl-4-yl}-isoxazol-4-yl)-carbamic acid (R)-1-phenyl-ethyl ester (Compound G); (3-Methyl-5-{4′-[1-(1H-tetrazol-5-yl)-cyclopropyl]-biphenyl-4-yl}-isoxazol-4-yl)-carbamic acid (R)-1-phenyl-ethyl ester (Compound H); or a pharmaceutically acceptable salt, prodrug, active metabolite, or a pharmaceutically acceptable solvate thereof.

In some embodiments, the LPA1 receptor antagonist is 6-(4-{4-[1-(2-Chloro-phenyl)-ethoxycarbonylamino]-3-methyl-isoxazol-5-yl}-phenyl)-hex-5-ynoic acid, or 7-(4-{4-[1-(2-chloro-phenyl)-ethoxycarbonylamino]-3-methyl-isoxazol-5-yl}-phenyl)-hept-6-ynoic acid, or a pharmaceutically acceptable salt, prodrug, active metabolite, or a pharmaceutically acceptable solvate thereof.

In some embodiments, the LPA1 receptor antagonist is systemically administered to the mammal.

In some embodiments, the LPA1 receptor antagonist is orally administered to the mammal.

In some embodiments, the LPA1 receptor antagonist is administered to the mammal in the form of an oral solution, oral suspension, powder, pill, tablet or capsule.

In some embodiments, the LPA1 receptor antagonist is topically administered to the eye of the mammal.

In some embodiments, the LPA1 receptor antagonist is topically administered to the eye of the mammal in the form of a solution, suspension, emulsion, ointment, cream, lotion, gel, colloidal dispersion, spray, drop, or combinations thereof.

In some embodiments, the ophthalmic formulation is administered via implantation, insertion, injection, spraying, washing, or combinations thereof.

In some embodiments, the methods of treatment further comprise administering to the mammal a therapeutically-effective amount of an second agent selected from antibiotics; anti-fungal agents; steroid anti-inflammatory agents; non-steroidal anti-inflammatory agents; antihistamines; antivirals; alpha agonists; beta blockers; carbonic anhydrase inhibitors; miotics; prostaglandins; anti-angiogenesis agents; loteprednol etabonate; mast cell stabilizers; cyclosporine; leukotriene synthesis inhibitors and DP2 receptor antagonists.

In some embodiments, described is an ophthalmic formulation comprising an LPA1 receptor antagonist and at least one suitable pharmaceutically acceptable excipient, wherein the formulation is in a form suitable for administration to the eye of a mammal. In some embodiments, the LPA1 receptor antagonist is in an amount effective for the treatment of an ophthalmic disease or condition in a mammal. In some embodiments, the ophthalmic disease or condition is a disease or condition described herein. In some embodiments, the ophthalmic formulation is in the form of a solution, suspension, emulsion, ointment, cream, lotion, gel, colloidal dispersion, or spray. In some embodiments, the LPA1 receptor antagonist is in an amount effective for the treatment of an ophthalmic disease or condition in a mammal, and the ophthalmic formulation is in the form of a solution, suspension, emulsion, ointment, cream, lotion, gel, colloidal dispersion, or spray.

In some embodiments, described is an ophthalmic formulation comprising an LPA1 receptor antagonist in an amount effective for the treatment of an ophthalmic disease or condition in a mammal, and at least one suitable pharmaceutically acceptable excipient to provide a solution, suspension, emulsion, ointment, cream, lotion, gel, colloidal dispersion, or spray.

In some embodiments, the pharmaceutically acceptable excipient is selected from pH adjusting agents, cosolvents, emulsifiers, penetration enhancers, preservatives, emollients, and combinations thereof.

In some embodiments, the ophthalmic formulation further comprises a suitable tonicity adjusting component. In some embodiments, the tonicity adjusting component is sodium borate, boric acid, sodium chloride, potassium chloride, mannitol, dextrose, glycerin, propylene glycol or mixtures thereof.

In some embodiments, the ophthalmic formulation is in the form of a solution that is administered to the mammal in the form of an eye drop.

In some embodiments, the ophthalmic formulation is in the form of a solution that is administered to the mammal in the form of an eye ointment.

In some embodiments, the concentration of the LPA1 receptor antagonist is about 0.1 to about 20% by weight of the formulation.

In some embodiments, the ocular disease or condition is LPA-dependent or LPA-mediated.

In some embodiments, the ocular disease or condition is LPA1-dependent or LPA1-mediated.

In some embodiments, the ocular disease or condition is an ocular disease or condition affecting the posterior segment of the eye, the anterior segment of the eye, or both the posterior segment of the eye and the anterior segment of the eye.

In some embodiments, the ocular disease or condition is an ocular disease or condition affecting the posterior segment of the eye.

In some embodiments, the ocular disease or condition is dry age related macular degeneration, geographic atrophy, wet age related macular degeneration, wet age related macular degeneration with neovascularization, wet age related macular degeneration with foveal thickening, diabetic retinopathy, diabetic retinopathy with retinal edema, diabetic retinopathy with neovascularization, retinitis pigmentosa, retinal degenerative diseases, proliferative vitreoretinopathy (PVR), prevention and treatment of macular thickening related to photocoagulation, retinopathy of prematurity (ROP), retinal detachment, post-surgical macular edema, posterior uveitis, macular edema associated with inherited retinal disease, chronic retinal macular edema, Usher syndrome, Bardet-Biedl syndrome (BBS), branch retinal vein occlusion (BRVO), or central retinal vein occlusion (CRVO).

In some embodiments, the ocular disease or condition is an ocular disease or condition affecting the anterior segment of the eye.

In some embodiments, the ocular disease or condition is ocular hypertension, primary open-angle glaucoma, episcleral fibrosis leading to trabeculectormy (bleb), failure after glaucoma filtration surgery, dry eyes, Sjogren syndrome, inflammation following ocular surgery, keratoconjuctivitis, pterygia, non-specific orbital inflammation, cataracts, post-surgical corneal scarring, corneal scarring, scarring associated with ocular cicatricial pemphigoid, glaucoma filtration surgery, thyroid eye disease, anterior uveitis, or fibrosis associated with keratoprosthesis procedure.

In some embodiments, the ophthalmic formulation further comprises a therapeutically-effective amount of an second agent selected from antibiotics; anti-fungal agents; steroid anti-inflammatory agents; non-steroidal anti-inflammatory agents; antihistamines; antivirals; alpha agonists; beta blockers; carbonic anhydrase inhibitors; miotics; prostaglandins; anti-angiogenesis agents; loteprednol etabonate; mast cell stabilizers; cyclosporine; leukotriene synthesis inhibitors and DP2 receptor antagonists.

In some embodiments, described herein, in some embodiments, are topical ophthalmic formulations for treating LPA-dependent or LPA-mediated diseases or conditions. The formulations described herein are suitable for ocular administration. The ophthalmic formulations described herein include one or more LPA receptor antagonists and allow for rapid delivery of a therapeutically effective amount of an LPA receptor antagonist into the circulatory system and/or target organ (e.g., the eye) of a mammal in need thereof. Administration of an ophthalmic formulation described herein to eyes of a mammal reverses, ameliorates, treats or prevents diseases or conditions in which the physiological activity of LPA is involved in the etiology or pathology of a disease or condition, or is otherwise associated with at least one symptom of a disease or condition.

In some embodiments, provided herein are ophthalmic formulations comprising an LPA1 receptor antagonist in an amount effective for the treatment of an LPA-dependent or LPA-mediated disease or condition, and at least one pharmaceutically acceptable excipient to provide a solution, suspension, emulsion, gel, ointment, drops, or an insert, wherein the formulation is in a form suitable for administration to the eyes of a mammal.

Also provided herein are ophthalmic formulations comprising an LPA1 receptor antagonist in an amount effective for antagonizing LPA receptors, and at least one pharmaceutically acceptable excipient to provide a solution, suspension, emulsion, gel, ointment, drops, or an insert, wherein the formulation is in a form suitable for administration to the eyes of a mammal.

In some embodiments, the LPA-dependent or LPA-mediated disease or condition is eye cancer, proliferative vitreoretinopathy, radiation induced corneal scarring, laser-assisted in situ keratomileusis, corneal transplant related disease or condition, trabeculectomy related disease or condition, ocular fibrosis, corneal ulcers, dry eye, keratoconjunctivisits sicca, age-related macular degeneration, allergic conjunctivitis, anterior segment scarring, blepharitis, blepharoconjunctivitis, cicatricial pemphigoid, conjunctival melanoma, conjunctivitis, contact lens-associated giant papillary conjunctivitis, diabetic retinopathy, episcleritis, glaucoma, reticular gliosis, Graves' ophthalmopathy, intraocular melanoma, keratitis, pain, pinguecula, post-surgical pain, pterygia, scarring, scleritis, Sjögren's syndrome, uveitis, vernal keratoconjunctivitis or combinations thereof.

In some embodiments, the LPA-dependent or LPA-mediated disease or condition is radiation induced corneal scarring. In some embodiments, the LPA-dependent or LPA-mediated disease or condition is keratoconjunctivitis. In some embodiments, the LPA-dependent or LPA-mediated disease or condition is corneal transplant related disease or condition. In some embodiments, the LPA-dependent or LPA-mediated disease or condition is trabeculectomy related disease or condition. In some embodiments, the LPA-dependent or LPA-mediated disease or condition is cicatricial pemphigoid. In some embodiments, the LPA-dependent or LPA-mediated disease or condition is intraocular melanoma. In some embodiments, the LPA-dependent or LPA-mediated disease or condition is proliferative vitreoretinopathy. In some embodiments, the LPA-dependent or LPA-mediated disease or condition is pterygia. In some embodiments, the LPA-dependent or LPA-mediated disease or condition scleritis.

In some embodiments, an ophthalmic formulation comprises an LPA1 receptor antagonist that has a structure of Formula (I), Formula (II), Formula (III), Formula (IV), Formula (V), Formula (VI) or Formula (VII), or a pharmaceutically acceptable salt thereof. In some embodiments, an ophthalmic formulation comprises an LPA1 receptor antagonist that has a structure of Formula (I), or a pharmaceutically acceptable salt thereof. In some embodiments, an ophthalmic formulation comprises an LPA1 receptor antagonist that has a structure of Formula (II), or a pharmaceutically acceptable salt thereof. In some embodiments, an ophthalmic formulation comprises an LPA1 receptor antagonist that has a structure of Formula (III), or a pharmaceutically acceptable salt thereof. In some embodiments, an ophthalmic formulation comprises an LPA1 receptor antagonist that has a structure of Formula (IV), or a pharmaceutically acceptable salt thereof. In some embodiments, an ophthalmic formulation comprises an LPA1 receptor antagonist that has a structure of Formula (V), or a pharmaceutically acceptable salt thereof. In some embodiments, an ophthalmic formulation comprises an LPA1 receptor antagonist that has a structure of Formula (VI) or a pharmaceutically acceptable salt thereof. In some embodiments, an ophthalmic formulation comprises an LPA1 receptor antagonist that has a structure of Formula (VI), or a pharmaceutically acceptable salt thereof.

In some embodiments, an ophthalmic formulation comprises an LPA receptor antagonist selected from LPA1 receptor antagonists disclosed in: U.S. Provisional Application No. 61/122,568; U.S. Provisional Application No. 61/183,785; U.S. patent application Ser. No. 12/638,702; U.S. Provisional Application No. 61/121,862; U.S. Provisional Application No. 61/231,282; U.S. Provisional Application No. 61/247,681; U.S. Provisional Application No. 61/247,2877; International patent application no. PCT/US2010/44284; International patent application no. PCT/US2010/51199; International patent application no. PCT/US2010/51150; U.S. patent application Ser. No. 12/896,080; International patent application no. PCT/US2010/50786; International patent application no. PCT/US2010/50787; U.S. patent application Ser. No. 12/893,902; International patent application no. PCT/US09/68106; International patent application no. PCT/US09/68105; International patent application no. PCT/US09/67527; International patent application no. PCT/US10/37309; International patent application no. PCT/US10/37316; or U.S. patent application Ser. No. 12/793,440; each of which is herein incorporated by reference.

Also provided herein is a method of treating of an LPA-dependent or LPA-mediated disease or condition, comprising administering to a mammal in need thereof a therapeutically-effective amount of an ophthalmic formulation described herein.

Also provided herein is a method of antagonizing ocular LPA receptors in a mammal in need thereof, comprising administering to the mammal a therapeutically-effective amount of an ophthalmic formulation described herein.

In certain embodiments, the LPA-dependent or LPA-mediated disease or condition is eye cancer, proliferative vitreoretinopathy, radiation induced corneal scarring, laser-assisted in situ keratomileusis, corneal transplant related disease or condition, trabeculectomy related disease or condition, ocular fibrosis, corneal ulcers, dry eye, keratoconjunctivisits sicca, age-related macular degeneration, allergic conjunctivitis, anterior segment scarring, blepharitis, blepharoconjunctivitis, cicatricial pemphigoid, conjunctival melanoma, conjunctivitis, contact lens-associated giant papillary conjunctivitis, diabetic retinopathy, episcleritis, glaucoma, reticular gliosis, Graves' ophthalmopathy, intraocular melanoma, keratitis, pain, pinguecula, post-surgical pain, pterygia, scarring, scleritis, Sjögren's syndrome, uveitis, vernal keratoconjunctivitis or combinations thereof.

In certain embodiments of the method, the LPA-dependent or LPA-mediated disease or condition is radiation induced corneal scarring. In some embodiments of the method, the LPA-dependent or LPA-mediated disease or condition is keratoconjunctivitis. In some embodiments of the method, the LPA-dependent or LPA-mediated disease or condition is corneal transplant related disease or condition. In some embodiments of the method, the LPA-dependent or LPA-mediated disease or condition is trabeculectomy related disease or condition. In some embodiments of the method, the LPA-dependent or LPA-mediated disease or condition is cicatricial pemphigoid. In some embodiments of the method, the LPA-dependent or LPA-mediated disease or condition is intraocular melanoma. In some embodiments of the method, the LPA-dependent or LPA-mediated disease or condition is proliferative vitreoretinopathy. In some embodiments of the method, the LPA-dependent or LPA-mediated disease or condition is pterygia. In some embodiments of the method, the LPA-dependent or LPA-mediated disease or condition is scleritis.

In some embodiments, the ophthalmic formulation is in the form of a solution, suspension, emulsion, emulsion, colloidal dispersion, spray, dry powder, aerosol, or drops, or combinations thereof.

In some embodiments, the formulation is administered as an eye drop. In some embodiments, the formulation is administered as an eye ointment.

In some embodiments, the concentration of the LPA1 receptor antagonist is about 0.1 to about 10% by weight of the formulation. In some embodiments, the concentration of the LPA receptor antagonist is about 0.25 to about 5% by weight of the formulation.

Provided herein is a method of increasing the concentrations of an LPA receptor antagonist in the eyes of a mammal comprising administering to a mammal in need thereof a therapeutically effective amount of an ophthalmic formulation described herein.

In certain embodiments, the mammal has at least one symptom of an LPA-dependent or LPA-mediated disease or condition affecting the eyes. In some embodiments, the mammal has at least one symptom of an LPA₁-dependent or LPA₁-mediated disease or condition affecting the eyes.

In some embodiments, the ophthalmic formulations provided herein are used to antagonize at least one LPA receptor in the eyes of a mammal in need thereof. In some embodiments, ophthalmic formulations provided herein are used to antagonize at least one LPA receptor for the treatment of a disease or condition that would benefit from antagonizing at least one LPA receptor in the eyes of a mammal in need thereof. In one aspect, the LPA receptor antagonized is LPA₁. In one aspect the LPA receptors antagonized are LPA₁ and LPA₃ receptors.

Articles of manufacture, which include packaging material, ophthalmic formulations within the packaging material, and a label that indicates that the compound or composition, or pharmaceutically acceptable salt, tautomers, pharmaceutically acceptable N-oxide, pharmaceutically active metabolite, pharmaceutically acceptable prodrug, or pharmaceutically acceptable solvate thereof, used for inhibiting the activity of at least one LPA receptor, or for the treatment, prevention or amelioration of one or more symptoms of a disease or condition that would benefit from inhibition of the activity of at least one LPA receptor, are provided.

Other objects, features and advantages will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

Lysophospholipids are membrane-derived bioactive lipid mediators. Lysophospholipids include, but are not limited to, lysophosphatidic acid (1-acyl-2-hydroxy-sn-glycero-3-phosphate; LPA), sphingosine 1-phosphate (SIP), lysophosphatidylcholine (LPC), and sphingosylphosphorylcholine (SPC). The lysophospholipid LPA acts through sets of specific G protein-coupled receptors (GPCRs) in an autocrine and paracrine fashion.

LPA binding to its cognate GPCRs (LPA₁, LPA₂, LPA₃, LPA₄, LPA₅, LPA₆) activates intracellular signaling pathways that mediate a variety of biological responses, including e.g., beneficial processes such as wound healing, angiogenesis, myelination, immunity and/or neurogenesis. LPA binding to its cognate GPCRs (LPA₁, LPA₂, LPA₃, LPA₄, LPA₅, LPA₆) also plays a role in physiological pathways related to inflammation, proliferative conditions, and/or carcinogenesis.

The cellular responses to LPA are predominantly mediated through lysophosphatidic acid receptors. Thus, LPA binding to LPA receptors mediates the pathology of disorders, diseases or conditions associated with, for example, aberrant wound healing, cell proliferation, block in apoptosis, and/or inflammation. LPA receptor antagonists disrupt LPA-dependent or LPA-mediated biological processes and reverse, ameliorate, prevent and/or treat LPA-dependent or LPA-mediated diseases or conditions.

In one aspect, disclosed herein is the use of LPA receptor antagonists in the treatment or prevention of LPA-dependent or LPA-mediated diseases, disorders or conditions associated with the eyes of a mammal. Disclosed herein is the use of LPA receptor antagonists in the manufacture of medicaments suitable for administration to the eyes of a mammal for the treatment or prevention of LPA-dependent or LPA-mediated diseases, disorders or conditions. In one aspect, the LPA receptor antagonists are LPA₁ receptor antagonists. In another aspect, the LPA receptor antagonists are dual antagonists of LPA₁ and LPA₃ receptors.

In some embodiments, an LPA receptor antagonist is administered orally or parenterally, wherein the LPA receptor antagonist reaches target tissues in the eye through systemic distribution.

In certain embodiments, an LPA receptor antagonist is administered locally to the eye of a mammal. In some embodiments, localized antagonism of LPA receptors in ophthalmic tissues by-passes gastrointestinal side-effects caused by systemic administration. In addition, localized antagonism of LPA receptors in the eyes of an individual avoids attenuation of beneficial effects of LPA in other parts of the body of the individual.

Described herein, in some embodiments, are ophthalmic formulations for administration to the eyes of a mammal that include an LPA receptor antagonist compound for treating an LPA-dependent or LPA-mediated disease, disorder or condition. In certain embodiments, ophthalmic formulations and compositions of LPA receptor antagonist compounds for the treatment of a LPA-dependent or LPA-mediated diseases, disorders, or conditions are delivered to the eyes of a mammal by (e.g., intravitreal or subtenon) injections. In some embodiments, ophthalmic formulations and compositions of LPA receptor antagonist compounds for the treatment of a LPA-dependent or LPA-mediated diseases, disorders, or conditions are delivered to the eyes of a mammal using ocular implants or devices.

In one aspect, ocular administration of an LPA receptor antagonist compound minimizes systemic absorption of the LPA receptor antagonist compound. In another aspect, treatment of LPA-dependent or LPA-mediated diseases, disorders, or conditions with an ophthalmic formulation described herein minimizes systemic absorption of an LPA receptor antagonist compound.

### LPA-Mediated or LPA-Dependent Diseases or Conditions

Normal wound healing occurs by a highly coordinated sequence of events in which cellular, soluble factors and matrix components act in concert to repair the injury. Activated platelets play pivotal roles in wound healing processes by releasing bioactive mediators to induce cell proliferation, cell migration, blood coagulation, and angiogenesis. LPA is one such mediator that is released from activated platelets; this induces platelet aggregation along with mitogenic/migration effects on the surrounding cells, such as endothelial cells, smooth muscle cells, fibroblasts, and keratinocytes.

LPA regulates many important functions of fibroblasts in wound healing, including proliferation, migration, differentiation and contraction. Fibroblast proliferation is required in wound healing in order to fill an open wound. In contrast, fibrosis is characterized by intense proliferation and accumulation of myofibroblasts that actively synthesize ECM and proinflammatory cytokines. LPA can either increase or suppress the proliferation of cell types important in wound healing, such as epithelial and endothelial cells (EC), macrophages, keratinocytes, and fibroblasts. A role for LPA₁ in LPA-induced proliferation was provided by the observation that LPA-stimulated proliferation of fibroblasts isolated from LPA₁ receptor null mice was attenuated (Mills et al, Nat Rev. Cancer 2003; 3: 582-591). LPA induces cytoskeletal changes that are integral to fibroblast adhesion, migration, differentiation and contraction.

Tissue injury initiates a complex series of host wound-healing responses; if successful, these responses restore normal tissue structure and function. Aberrant responses can lead to tissue fibrosis, inflammation, collagen deposition and/or proliferation.

### Ocular Fibrosis

LPA is involved in wound healing in the eye. LPA₁ and LPA₃ receptors are detectable in the normal rabbit corneal epithelial cells, keratocytes and lens epithelial cells and LPA₁ and LPA₃ expression are increased in corneal epithelial cells following injury. LPA and its homologues are present in the aqueous humor and the lacrimal gland fluid of the rabbit eye following corneal injury and these levels are increased in a rabbit corneal injury model.

LPA induces actin stress fiber formation in rabbit corneal endothelial and epithelial cells and promotes contraction of corneal fibroblasts. Thus LPA contributes to a wound healing response in ocular tissues.

In some instances, corneal scarring is caused by injury to the cornea (abrasion, laceration, burns, or disease). Surface abrasions heal transparently and do not leave scars. Deeper abrasions and ulcerations/lacerations result in a loss of corneal tissue, which is replaced by scar tissue. Proliferation of new blood vessels in the clear cornea assists in the healing process. Aberrant wound healing results in loss of vision.

In certain instances, fluid from the vitreous humor enters a retinal hole during rhegmatogenous retinal detachment. The accumulation of fluid in the subretinal space and the tractional force of the vitreous on the retina result in rhegmatogenous retinal detachment. During this process the retinal cell layers come in contact with vitreous cytokines. These cytokines trigger the ability of the retinal pigmented epithelium (RPE) to proliferate and migrate. The process involved resembles fibrotic wound healing by the RPE cells. The RPE cells undergo epithelial-mesenchymal transition (EMT), giving them the ability to migrate out into the vitreous, proliferate excessively and lay down extracellular matrix (ECM) on both side sides of the detached retina. The two membranes differ in composition in that the epiretinal membrane is composed of RPE cells, glial cells, macrophages and fibrocytes, while the subretinal membrane is rich in RPE cells. During this process the RPE cell layer-neural retinal adhesion and RPE-ECM (extracellular matrix) adhesions are lost. The RPE cells lay down fibrotic membranes while they migrate and these membranes contract and pull at the retina. All of these processes finally lead to secondary retinal detachment (proliferative vitreoretinopathy (PVR)) after primary retinal detachment surgery. In some instances, LPA induces RPE cell proliferation and thus plays a role in the development of PVR.

In one aspect, LPA receptor antagonists are used in the treatment of various fibroses associated with LPA-mediated or LPA-dependent aberrant wound healing and/or fibrosis in a mammal. In some embodiments, LPA receptor antagonists are administered to a mammal and antagonize LPA receptors in the eyes of the mammal. In certain embodiments, LPA receptor antagonists are administered to a mammal and antagonize LPA₁ receptors in the eye. In some instances, antagonizing LPA receptors associated with aberrant wound healing and/or fibrosis reduces or inhibits the proliferation of fibroblasts and/or increases apoptosis of fibroblasts associated with fibrotic disorders, inflammation and/or proliferative disorders of the eye. In one aspect, LPA receptor antagonists reduce, ameliorate or inhibit aberrant wound healing, fibroblast proliferation and/or fibrosis associated with LPA-dependent or LPA-mediated fibrotic disorders. In another aspect, LPA receptor antagonists reduce, ameliorate or inhibit fibrosis and/or aberrant wound healing in ocular tissues. In some embodiments, the LPA receptors are LPA₁ and/or LPA₃ receptors. In certain embodiments, the LPA receptors are LPA₁ receptors.

In another aspect, LPA receptor antagonists are used to improve the corneal sensitivity decrease caused by corneal operations such as laser-assisted in situ keratomileusis (LASIK) or cataract operation, corneal sensitivity decrease caused by corneal degeneration, and dry eye symptom caused thereby.

In yet another aspect, LPA receptor antagonists are used to reduce, ameliorate, or inhibit aberrant wound healing and/or scarring of ocular tissues (e.g., the cornea or retina). In some instances, scarring is the result of disease e.g., keratitis (e.g., inflammation caused by herpes simplex, or syphilis). In some instances, surgical procedures including, for example, corneal graft, corneal transplant, trabeculectomy and/or radiation assisted eye surgery induce corneal scarring. In certain instances, corneal injury is due to laser assisted in situ keratomileusis (LASIK). In some instances, corneal scarring is due to corneal ulcers. In certain instances, proliferative membranes of the posterior segment of the eye induce the deposition of mutilating fibrous tissue and consequent production of scar tissue. In some instances, retinal thinning and scarring is due to structural changes in the retina caused by chronic cystoid macular edema (chronic CME). In certain instances, disorganized growth of retinal blood vessels in prematurely born babies results in scarring and/or retinal detachment (retinopathy of prematurity (ROP)) In some instances, bleeding, leaking and scarring from abnormal blood vessel growth (choroidal neovascularization) due to wet age related macular degeneration (wet AMD) causes irreversible damage to photoreceptors and rapid vision loss if left untreated.

Examples of disorders associated with aberrant wound healing and/or scarring of ocular tissues that, in some embodiments, are treated with LPA receptor antagonists are episcleral fibrosis leading to bleb (trabeculectomy) failure after glaucoma filtration surgery, pterygia (including post-surgical wound healing/scarring), cataracts (post surgical scarring), corneal scarring, scarring associated with ocular cicatricial pemphigoid, glaucoma filtration surgery (trabeculectomy), fibrosis associated with a keratoprosthesis procedure, wet age related macular degeneration with neovascularization and foveal thickening, diabetic retinopathy with retinal edema (and neovascularization), proliferative vitreoretinopathy (PVR), prevention and treatment of macular thickening related to photocoagulation, retinopathy of prematurity (ROP), (primary) retinal detachment, chronic retinal macular edema, chronic cystoid macular edema, post-surgical macular edema, macular edema associated with inherited retinal disease.

### Inflammation

LPA has been shown to regulate immunological responses by modulating activities/functions of immune cells such as T-/B-lymphocytes and macrophages. In activated T cells, LPA activates IL-2 production/cell proliferation through LPA₁ (Gardell et al, TRENDS in Molecular Medicine Vol. 12 No. 2 February 2006). Expression of LPA-induced inflammatory response genes is mediated by LPA₁ and LPA₃ (Biochem Biophys Res Commun. 363(4):1001-8, 2007). In addition, LPA modulates the chemotaxis of inflammatory cells (Biochem Biophys Res Commun., 1993, 15; 193(2), 497). The proliferation and cytokine-secreting activity in response to LPA of immune cells (J. Imuunol. 1999, 162, 2049), platelet aggregation activity in response to LPA, acceleration of migration activity in monocytes, activation of NF-κB in fibroblast, enhancement of fibronectin-binding to the cell surface, and the like are known. Thus, LPA is associated with various inflammatory/immune diseases.

In one aspect, LPA receptor antagonists are used to treat or prevent inflammation of tissues of the eye of mammal. In another aspect, antagonists of LPA₁ and/or LPA₃ find use in the treatment or prevention of inflammatory/immune disorders affecting the eye of a mammal. In yet another aspect, the LPA receptor antagonist used to treat or prevent inflammation of tissues of the eye of a mammal is a LPA₁ receptor antagonist.

In a further aspect, presented herein is the use of an LPA receptor antagonist in the treatment or prevention of ocular inflammation, vernal keratoconjunctivitis, and papillary conjunctivitis in a mammal comprising administering at least once to the mammal an effective amount of at least one LPA receptor antagonist.

In yet another aspect, presented herein is the use of an LPA receptor antagonist in the treatment or prevention of Sjögren disease or inflammatory disease with dry eyes in a mammal comprising administering at least once to the mammal an effective amount of at least one LPA receptor antagonist.

Examples of inflammatory/immune disorders affecting the eyes of a mammal that, in some embodiments, are treated with LPA receptor antagonists include ocular hypertension, primary open-angle glaucoma, dry eyes (keratoconjunctivitis sicca), Sjögren's syndrome, inflammation following ocular surgery, keratoconjunctivitis, anterior and posterior uveitis, idiopathic orbital inflammation (non-specific orbital inflammation), ocular cicatricial pemphigoid, thyroid eye disease (Graves' ophthalmopathy), post-surgical macular edema, macular edema associated with inherited retinal disease.

### Proliferative and Inflammatory Conditions

Lysophosphatidic acid (LPA) and its G protein-coupled receptors (GPCRs) LPA₁, LPA₂, and/or LPA₃ play a role in the manifestation of inflammation. In the eye, LPA serves as an inflammatory mediator in human corneal epithelial cells. Corneal epithelial cells generate LPA in response to inflammatory stimuli and LPA elicits diverse biological actions including proliferation, chemotaxis, cytokine secretion and/or activation of Erk, Akt, p38 or the like.

LPA secreted from corneal epithelial cells is present in the tear film, providing an interface between aqueous and non-polar lipid layers as well as acting as a proinflammatory agent. Inflammatory changes in the ocular surface cause disorders such as, by way of example, dry eye (keratoconjunctivisits sicca). Dry eye causes histological changes in the ocular surface epithelium including aberrant proliferation and cell differentiation. LPA-induced pro-inflammatory cytokines in the tears contribute to the pathology of ocular diseases that are caused by persistent inflammation. Inflammation in the lacrimal glands leads to dry eye (e.g., dry eye due to Sjögren's syndrome).

The interrelationship between inflammation and proliferation is observed in several pathological settings including, for example, cancer. Lysophospholipid receptor signaling plays a role in the etiology of cancer. Lysophosphatidic acid (LPA) and its G protein-coupled receptors (GPCRs) LPA₁, LPA₂, and/or LPA₃ play a role in the development of several types of cancers. The initiation, progression and metastasis of cancer involve several concurrent and sequential processes including cell proliferation and growth, survival and anti-apoptosis, migration of cells, penetration of foreign cells into defined cellular layers and/or organs, and promotion of angiogenesis.

LPA signals through its own GPCRs leading to activation of multiple downstream effector pathways. Such downstream effector pathways play a role in cancer. LPA and its GPCRs are linked to cancer through major oncogenic signaling pathways. LPA contributes to tumorigenesis by increasing motility and invasiveness of cells. LPA protects epithelial and fibroblast cell lines from apoptosis. The suppression of the p53 transcription factor by LPA stimulates cancer cell division, reduces apoptosis, and thereby promotes tumor progression.

LPA also induces proliferation of human retinal pigmented epithelial (RPE) cells. Proliferative vitreoretinopathy (PVR) arises from an exaggerated wound-healing response by RPE cells. Other ocular conditions associated with inflammation and/or proliferation include, for example, age-related macular degeneration, allergic conjunctivitis, blepharitis, blepharoconjunctivitis, cicatricial pemphigoid, conjunctival melanoma, conjunctivitis, contact lens-associated giant papillary conjunctivitis, diabetic retinopathy, episcleritis, glaucoma, reticular gliosis, Graves' ophthalmopathy, intraocular melanoma, keratitis, pain, pinguecula, post-surgical pain, pterygia, scleritis, Sjögren's syndrome, uveitis, vernal keratoconjunctivitis or combinations thereof.

In one aspect, the LPA receptor antagonists are used in the treatment of eye inflammation and/or eye cancers. In one aspect, the LPA receptor antagonists reduce, ameliorate or inhibit inflammation, cytokine secretion and/or proliferation in ocular tissues. In one aspect, the LPA receptor antagonists are administered to the eyes of a mammal and antagonize LPA receptors associated with cell proliferation. In some instances, antagonizing LPA receptors associated with cell proliferation reduces or inhibits the proliferation of fibroblasts or melanocytes or the like and/or increases apoptosis of fibroblasts, melanocytes and the like that are associated with proliferative disorders of the eye. In another aspect, the LPA receptor antagonists reduce, ameliorate or inhibit cytokine secretion and/or inflammation associated with LPA-dependent or LPA-mediated inflammatory conditions. In one aspect, the LPA receptors are LPA₁ and/or LPA₃ receptors.

Examples of eye disorders associated with inflammation and proliferation and treated, in some embodiments, with LPA receptor antagonists are dry age related macular degeneration (geography atrophy), wet age related macular degeneration with neovascularization and foveal thickening, retinitis pigmentosa (RP) and other retinal degenerative diseases (e.g., Usher syndrome, Bardet-Biedl syndrome (BBS)), (proliferative) diabetic retinopathy with retinal edema (and neovascularization), proliferative vitreoretinopathy (PVR), prevention and treatment of macular thickening related to photocoagulation, (primary) retinal detachment, branch retinal vein occlusion (BRVO), central retinal vein occlusion (CRVO).

### LPA1 Receptor Antagonists

In some embodiments, the LPA1 receptor antagonist is a small molecule compound (“compound”), peptide, polypeptides, a peptidomimetics, proteins, an antibody, antibody ligand binding domains, an aptamer, or an oligonucleotide.

In certain aspects, the activity of LPA₁ in a mammal is directly or indirectly modulated by the administration of (at least once) a therapeutically effective amount of an LPA receptor antagonist. Such modulation includes, but is not limited to, reducing and/or inhibiting the activity of LPA₁. In additional aspects, the activity of LPA in a mammal is directly or indirectly modulated, including reducing and/or inhibiting, by the administration of (at least once) a therapeutically effective amount of an LPA receptor antagonist. Such modulation includes, but is not limited to, reducing and/or inhibiting the activity of an LPA receptor. In one aspect, the LPA receptors are LPA₁ and/or LPA₃ receptors. In one aspect, the LPA receptors are LPA₁ and/or LPA₂ receptors

### Compounds

LPA₁ receptor antagonists are disclosed herein or in any one of the following: U.S. Provisional Application No. 61/122,568; U.S. Provisional Application No. 61/183,785; U.S. patent application Ser. No. 12/638,702; U.S. Provisional Application No. 61/121,862; U.S. Provisional Application No. 61/231,282; U.S. Provisional Application No. 61/247,681; U.S. Provisional Application No. 61/247,2877; International patent application no. PCT/US2010/44284; International patent application no. PCT/US2010/51199; International patent application no. PCT/US2010/51150; U.S. patent application Ser. No. 12/896,080; International patent application no. PCT/US2010/50786; International patent application no. PCT/US2010/50787; U.S. patent application Ser. No. 12/893,902; International patent application no. PCT/US09/68106; International patent application no. PCT/US09/68105; International patent application no. PCT/US09/67527; International patent application no. PCT/US10/37309; International patent application no. PCT/US10/37316; U.S. patent application Ser. No. 12/793,440; each of which is herein incorporated by reference.

In some embodiments, the LPA1 receptor antagonist has the structure of Formula (I):

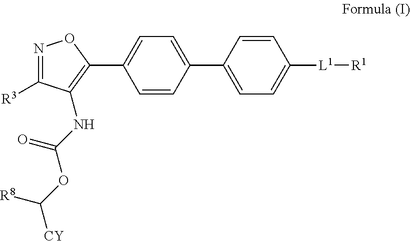

wherein,
R¹ is —CO₂H, —CO₂R^{D}, tetrazolyl, 5-oxo-2,5-dihydro-[1,2,4]oxadiazol-3-yl, —C(═O)NHSO₂CH₃, or —C(═O)NHSO₂CH₂CH₃; R^{D} is —CH₃, —CH₂CH₃, —CH₂CH₂CH₃, —CH(CH₃)₂, —CH₂CH₂CH₂CH₃, or —C(CH₃)₃;
L¹ is C₁-C₄alkylene or C₃-C₆cycloalkylene;
R³ is H, —CH₃, —CH₂CH₃, or —CF₃;
R⁸ is H or —CH₃;
CY is C₁-C₆alkyl, substituted or unsubstituted C₃-C₆cycloalkyl, or substituted or unsubstituted phenyl; wherein if CY is substituted then CY is substituted with 1 or 2 R^{C}; each R^{C} is independently F, Cl, Br, I, —OH, —CN, C₁-C₄alkyl, C₁-C₄fluoroalkyl, C₁-C₄fluoroalkoxy, or C₁-C₄alkoxy;
or a pharmaceutically acceptable salt, prodrug, active metabolite, or a pharmaceutically acceptable solvate thereof.

In some embodiments, CY is substituted or unsubstituted phenyl; wherein if CY is substituted then CY is substituted with 1 or 2 R^{C}; each R^{C} is independently F, Cl, —CN, —CH₃, —CF₃, —OH, —OCF₃, or —OCH₃. In some embodiments, CY is

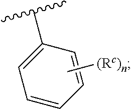

n is 0, 1, or 2. In some embodiments, n is 0 or 1. In some embodiments, CY is cyclopropyl, cyclobutyl, cyclopentyl, cyclopent-1-enyl, 2-chlorocyclopent-1-enyl, cyclohexyl, cyclohex-1-enyl, 2-chlorocyclohex-1-enyl, phenyl, 2-fluorophenyl, 2,3-difluorophenyl, 2,4-difluorophenyl, 2,5-difluorophenyl, 2,6-difluorophenyl, 2-chlorophenyl, 2,6-dichlorophenyl, 2-bromophenyl, 3-bromophenyl, 2,4-dichlorophenyl, 2-hydroxyphenyl, 3-hydroxyphenyl, 4-hydroxyphenyl, 2-methoxyphenyl, 3-methoxyphenyl, 4-methoxyphenyl, 2-trifluoromethylphenyl, 3-trifluoromethylphenyl, 4-trifluoromethylphenyl, 2-fluoro-4-methoxyphenyl, 2-methylphenyl, 3-methylphenyl, 4-methylphenyl, 2-cyanophenyl, 3-cyanophenyl, or 4-cyanophenyl. In some embodiments, CY is phenyl, 2-fluorophenyl, 2-chlorophenyl, 2-trifluoromethylphenyl, or 2-methylphenyl. In some embodiments, CY is phenyl, 2-fluorophenyl, or 2-chlorophenyl. In some embodiments, CY is phenyl.

In some embodiments, the compound of Formula (I) has the following structure:

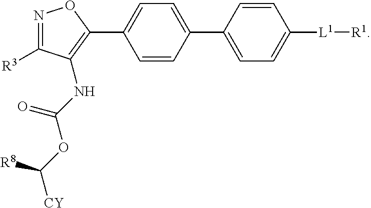

In some embodiments, the LPA receptor antagonist has the structure of Formula (II):

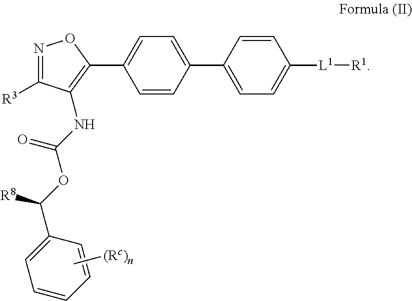

In some embodiments, R¹ is —CO₂H. In some embodiments, L¹ is —CH₂—, —CH₂CH₂—, —CH₂CH₂CH₂—, —CH(CH₃)—, —C(CH₃)₂—, —CH(CH₂CH₃)—, —C(CH₂CH₃)₂—, —CH₂CH(CH₃)—, —CH₂C(CH₃)₂—, cyclopropyl-1,1-diyl, cyclopropyl-1,2-diyl, cyclobutyl-1,1-diyl, cyclopentyl-1,1-diyl or cyclohexyl-1,1-diyl. In some embodiments, L¹ is —CH₂—, —C(CH₃)₂—, or —C(CH₂CH₃)₂—. In some embodiments, L¹ is —CH₂— or cyclopropyl-1,1-diyl. In some embodiments, L¹ is —CH₂—. In some embodiments, L¹ is cyclopropyl-1,1-diyl, cyclobutyl-1,1-diyl, cyclopentyl-1,1-diyl or cyclohexyl-1,1-diyl. In some embodiments, L¹ is cyclopropyl-1,1-diyl. In some embodiments, R³ is H. In some embodiments, R³ is —CH₃. In some embodiments, each of R^{C} is independently selected from F, Cl, —CH₃, and —CF₃; n is 0 or 1.

In some embodiments, the LPA1 receptor antagonist is:

### (R)-2-(4′-(3-methyl-4-((1-phenylethoxy)carbonylamino)isoxazol-5-yl)biphenyl-4-yl)acetic acid (Compound A)

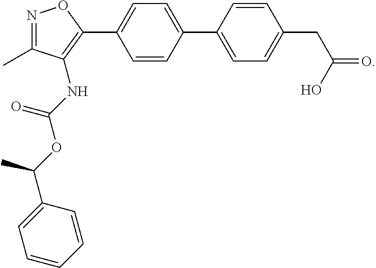 [SMILES: Cc1noc(-c2ccc(-c3ccc(CC(=O)O)cc3)cc2)c1NC(=O)O[C@H](C)c1ccccc1]

### (R)-1-(4′-(3-methyl-4-((1-phenylethoxy)carbonylamino)isoxazol-5-yl)biphenyl-4-yl)cyclopropanecarboxylic acid (Compound B)

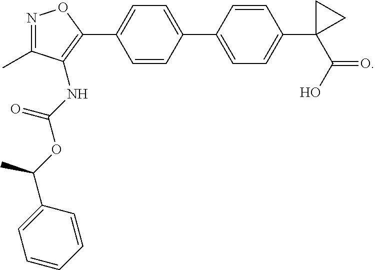 [SMILES: Cc1noc(-c2ccc(-c3ccc(C4(C(=O)O)CC4)cc3)cc2)c1NC(=O)O[C@H](C)c1ccccc1]

### (R)-2-(4′-(4-((1-(2-chlorophenyl)ethoxy)carbonylamino)-3-methylisoxazol-5-yl)biphenyl-4-yl)acetic acid (Compound C)

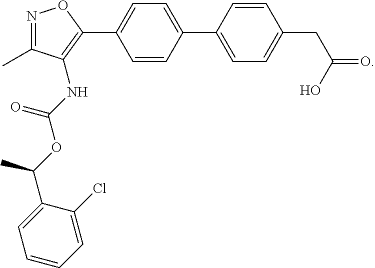 [SMILES: Cc1noc(-c2ccc(-c3ccc(CC(=O)O)cc3)cc2)c1NC(=O)O[C@H](C)c1ccccc1Cl]

### {5-[4′-(1-Methanesulfonylaminocarbonyl-cyclopropyl)-biphenyl-4-yl]-3-methyl-isoxazol-4-yl}-carbamic acid (R)-1-phenyl-ethyl ester (Compound D)

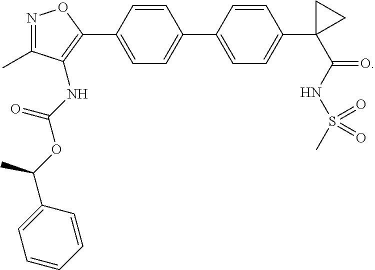 [SMILES: Cc1noc(-c2ccc(-c3ccc(C4(C(=O)NS(C)(=O)=O)CC4)cc3)cc2)c1NC(=O)O[C@H](C)c1ccccc1]

### 1-(4′-{4-[(R)-1-(2-Chloro-phenyl)-ethoxycarbonylamino]-3-methyl-isoxazol-5-yl}-biphenyl-4-yl)-cyclopropanecarboxylic acid (Compound E)

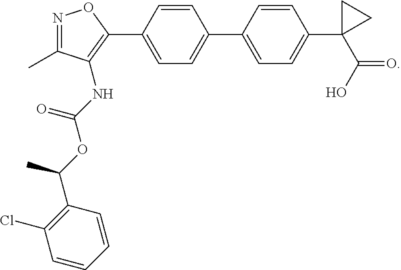 [SMILES: Cc1noc(-c2ccc(-c3ccc(C4(C(=O)O)CC4)cc3)cc2)c1NC(=O)O[C@H](C)c1ccccc1Cl]

### 1-{4′-[4-((R)-1-Phenyl-ethoxycarbonylamino)-isoxazol-5-yl]-biphenyl-4-yl}-cyclopropanecarboxylic acid (Compound F)

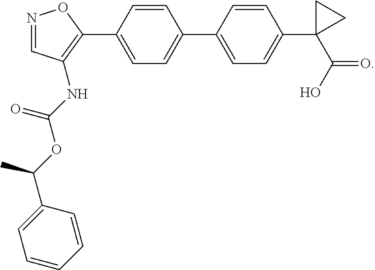 [SMILES: C[C@@H](OC(=O)Nc1cnoc1-c1ccc(-c2ccc(C3(C(=O)O)CC3)cc2)cc1)c1ccccc1]

### (3-Methyl-5-{4′-[1-(5-oxo-2,5-dihydro-[1,2,4]oxadiazol-3-yl)-cyclopropyl]-biphenyl-4-yl}-isoxazol-4-yl)-carbamic acid (R)-1-phenyl-ethyl ester (Compound G): or

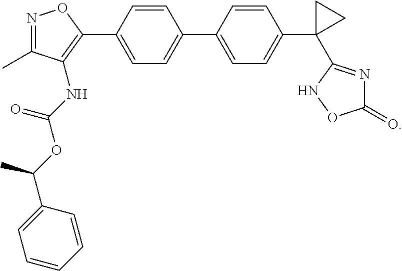 [SMILES: Cc1noc(-c2ccc(-c3ccc(C4(c5nc(=O)o[nH]5)CC4)cc3)cc2)c1NC(=O)O[C@H](C)c1ccccc1]

### (3-Methyl-5-{4′-[1-(1H-tetrazol-5-yl)-cyclopropyl]-biphenyl-4-yl}-isoxazol-4-yl)-carbamic acid (R)-1-phenyl-ethyl ester (Compound H)

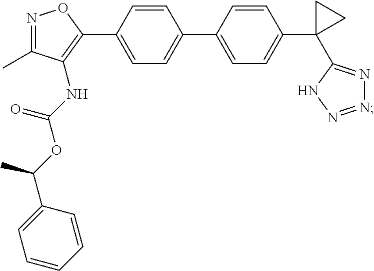 [SMILES: Cc1noc(-c2ccc(-c3ccc(C4(c5nnn[nH]5)CC4)cc3)cc2)c1NC(=O)O[C@H](C)c1ccccc1]

or a pharmaceutically acceptable salt, prodrug, active metabolite, or a pharmaceutically acceptable solvate thereof.

In some embodiments, the LPA1 receptor antagonist has structure of Formula (III):

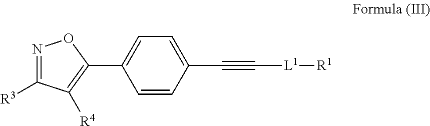

wherein,
R¹ is —CO₂H, —CO₂R^{D}, tetrazolyl, 5-oxo-2,5-dihydro-[1,2,4]oxadiazol-3-yl, —C(═O)NHSO₂CH₃, or —C(═O)NHSO₂CH₂CH₃; R^{D} is —CH₃, —CH₂CH₃, —CH₂CH₂CH₃, —CH(CH₃)₂, —CH₂CH₂CH₂CH₃, or —C(CH₃)₃;
L¹ is absent, or a C₁-C₆alkylene;
R³ is H, —CH₃, —CH₂CH₃, or —CF₃;
R⁴ is —NHC(═O)OCH(R⁸)—CY;
   R⁸ is H, or —CH₃;
   CY is substituted or unsubstituted phenyl; wherein if CY is substituted then CY is substituted with 1 or 2 R^{C}; each R^{C} is independently F, Cl, Br, I, —OH, —CN, C₁-C₄alkyl, C₁-C₄fluoroalkyl, C₁-C₄fluoroalkoxy, or C₁-C₄alkoxy;
or a pharmaceutically acceptable salt, prodrug, active metabolite, or a pharmaceutically acceptable solvate thereof.

In some embodiments, R¹ is —CO₂H or —CO₂R^{D}. In some embodiments, R¹ is —CO₂H. In some embodiments, L¹ is —CH₂—, —CH(CH₃)—, —C(CH₃)₂—, —CH₂CH₂—, —CH₂CH(CH₃)—, —CH₂C(CH₃)₂—, —CH₂CH₂CH₂—, or —CH₂CH₂CH₂CH₂—. In some embodiments, L¹ is —CH₂—, —CH₂CH₂—, —CH₂CH₂CH₂—, or —CH₂CH₂CH₂CH₂—. In some embodiments, L¹ is —CH₂CH₂CH₂—, or —CH₂CH₂CH₂CH₂—. In some embodiments, R³ is H. In some embodiments, R³ is —CH₃. In some embodiments, R⁸ is —CH₃. In some embodiments, each of R^{C} is independently selected from F, Cl, —CH₃, and —CF₃.

In some embodiments, R⁴ is

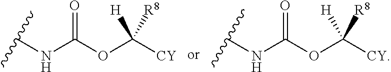

In some embodiments, CY is a substituted or unsubstituted phenyl, wherein if CY is substituted then CY is substituted with 1 or 2 R^{C}; each R^{C} is independently F, Cl, —CN, —CH₃, —CF₃, —OH, —OCF₃, or —OCH₃.

In some embodiments, the LPA1 antagonist is selected from: 6-(4-{4-[1-(2-Chloro-phenyl)-ethoxycarbonylamino]-3-methyl-isoxazol-5-yl}-phenyl)-hex-5-ynoic acid, 7-(4-{4-[1-(2-chloro-phenyl)-ethoxycarbonylamino]-3-methyl-isoxazol-5-yl}-phenyl)-hept-6-ynoic acid, or a pharmaceutically acceptable salt, prodrug, active metabolite, or a pharmaceutically acceptable solvate thereof.

In some embodiments, the LPA1 receptor antagonist has the structure of Formula (IV):

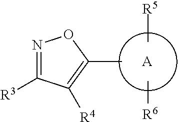

wherein,
A is an aryl or heteroaryl ring;
R³ is H, C₁-C₄alkyl, C₁-C₄fluoroalkyl;
R⁴ is —NHC(═O)OCH(R⁸)—CY, or —NHC(═O)O—CY;
   R⁸ is H, C₁-C₄alkyl, C₁-C₄fluoroalkyl;
   CY is a substituted or unsubstituted C₃-C₆cycloalkyl, a substituted or unsubstituted phenyl, or a substituted or unsubstituted monocyclic heteroaryl; wherein if CY is substituted then CY is substituted with 1 or 2 R^{C}; each R^{C} is independently selected from F, Cl, Br, I, —CN, —OH, C₁-C₄alkyl, C₁-C₄fluoroalkyl, C₁-C₄fluoroalkoxy, C₁-C₄alkoxy, and C₁-C₄heteroalkyl;
R⁵ and R⁶ are each independently selected from H, halogen, —CN, —NO₂, —OH, —OR¹⁰, C₁-C₄alkyl, C₁-C₄fluoroalkyl, C₁-C₄fluoroalkoxy, C₁-C₄alkoxy, and C₁-C₄heteroalkyl;
R¹⁰ is selected from C₁-C₆alkyl, C₁-C₆heteroalkyl, C₁-C₆fluoroalkyl, a substituted or unsubstituted cycloalkyl, a substituted or unsubstituted heterocycloalkyl;
or a pharmaceutically acceptable salt, prodrug, active metabolite, or a pharmaceutically acceptable solvate thereof.

In some embodiments, A is phenyl, or a 5- or 6-membered monocyclic heteroaryl. In some embodiments, A is a phenyl, pyridinyl, thiazolyl, or pyrimidinyl. In some embodiments, R⁵ and R⁶ are each independently selected from hydrogen, halogen, or hydroxy. In some embodiments, R³ is methyl, ethyl, isopropyl or trifluoromethyl. In some embodiments, R³ is methyl. In some embodiments, CY is a substituted or unsubstituted CY cycloalkyl, a substituted or unsubstituted heterocycloalkyl, a substituted or unsubstituted aryl, or a substituted or unsubstituted heteroaryl; or

In some embodiments, the LPA1 antagonist has one of the following structures:

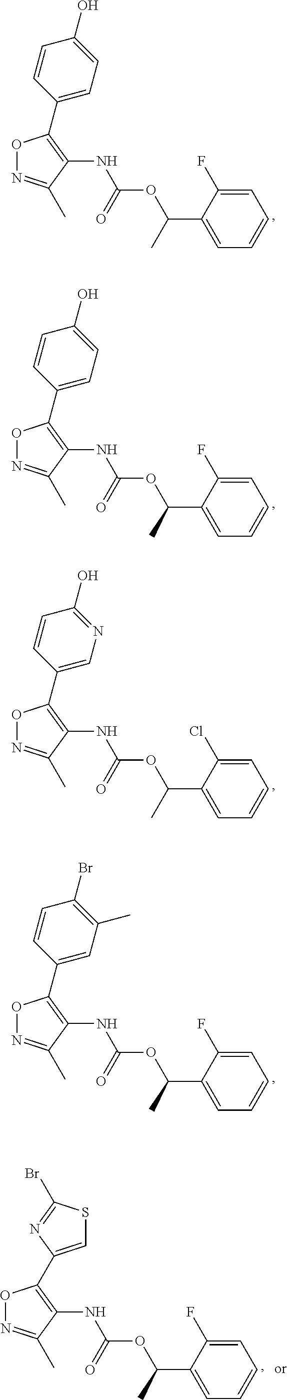

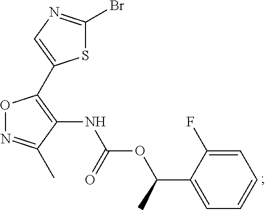 [SMILES: Cc1noc(-c2cnc(Br)s2)c1NC(=O)O[C@H](C)c1ccccc1F ; ";"]

or a pharmaceutically acceptable salt, prodrug, active metabolite, or a pharmaceutically acceptable solvate thereof.

In some embodiments, the LPA1 receptor antagonist has the structure of Formula (V):

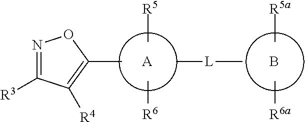

wherein,
A is an aryl or heteroaryl ring;
B is an aryl or heteroaryl ring;
L is absent, C₁-C₄alkylene, C₁-C₄heteroalkylene, —O—, —S—, —SO—, —SO₂—, —NH—, —NR²—, or —C(═O)—; R² is C₁-C₄alkyl;
R³ is H, C₁-C₄alkyl, or C₁-C₄fluoroalkyl;
R⁴ is —NHC(═O)OCH(R⁸)—CY, or —NHC(═O)O—CY;
   R⁸ is H, C₁-C₄alkyl, or C₁-C₄fluoroalkyl;
   CY is a substituted or unsubstituted cycloalkyl, a substituted or unsubstituted heterocycloalkyl, a substituted or unsubstituted aryl, or a substituted or unsubstituted heteroaryl; wherein if CY is substituted then CY is substituted with 1 or 2 R^{C}; each R^{C} is independently selected from F, Cl, Br, I, —CN, —OH, C₁-C₄alkyl, C₁-C₄fluoroalkyl, C₁-C₄fluoroalkoxy, C₁-C₄alkoxy, and C₁-C₄heteroalkyl;
R⁵ and R⁶ are each independently selected from H, halogen, —CN, —NO₂, —OH, —OR¹⁰, C₁-C₄alkyl, C₁-C₄fluoroalkyl, C₁-C₄fluoroalkoxy, C₁-C₄alkoxy, and C₁-C₄heteroalkyl;
R^{5a} and R^{6a} are each independently selected from H, halogen, —CN, —NO₂, —OH, —OR¹⁰, —S(═O)₂R¹⁰, substituted or unsubstituted C₁-C₄alkyl, C₁-C₄fluoroalkyl, C₁-C₄fluoroalkoxy, C₁-C₄alkoxy, C₁-C₄heteroalkyl, substituted or unsubstituted C₃-C₆cycloalkyl or substituted or unsubstituted C₁-C₆heterocycloalkyl;
R¹⁰ is selected from C₁-C₆alkyl, C₁-C₆heteroalkyl, C₁-C₆fluoroalkyl, a substituted or unsubstituted cycloalkyl, a substituted or unsubstituted heterocycloalkyl;
or a pharmaceutically acceptable salt, prodrug, active metabolite, or a pharmaceutically acceptable solvate thereof.

In some embodiments, ring A is a substituted or unsubstituted monocyclic ring wherein the groups

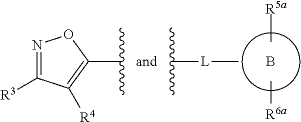

are in a 1,3-relationship on ring A (i.e. an meta relationship).

In some embodiments, ring A is a substituted or unsubstituted monocyclic ring wherein the groups

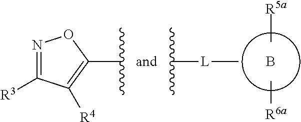

are in a 1,4-relationship on ring A (i.e. an para relationship).

In some embodiments, A is a phenyl ring. In some embodiments, A is a monocyclic heteroaryl. In some embodiments, A is a 6-membered monocyclic heteroaryl. In some embodiments, A is a pyridinyl ring. In some embodiments, L is absent. In some embodiments, L is absent and A-L-B is bi-aromatic. In some embodiments, L is absent and A-L-B is biphenyl. In some embodiments, B is a phenyl ring. In some embodiments, B is a monocyclic heteroaryl. In some embodiments, B is a 6-membered monocyclic heteroaryl. In some embodiments, B is a pyridinyl ring. In some embodiments, A-L-B is phenyl-pyridyl. In some embodiments, R⁵ and R⁶ are each independently selected from hydrogen, halogen, or hydroxy. In some embodiments, Rya and R^{6a} are each independently selected from hydrogen, halogen, hydroxy, hydroxymethyl or substituted or unsubstituted heterocycloalkyl. In some embodiments, R³ is methyl, ethyl, isopropyl or trifluoromethyl.

In some embodiments, L is absent, —CH₂—, —CH₂O—, —OCH₂—, —CH₂S—, —SCH₂—, —CH₂NH—, —NHCH₂—, —O—, —S—, or —NH—. In some embodiments, L² is absent.

In some embodiments, the LPA1 antagonist has a structure selected from:

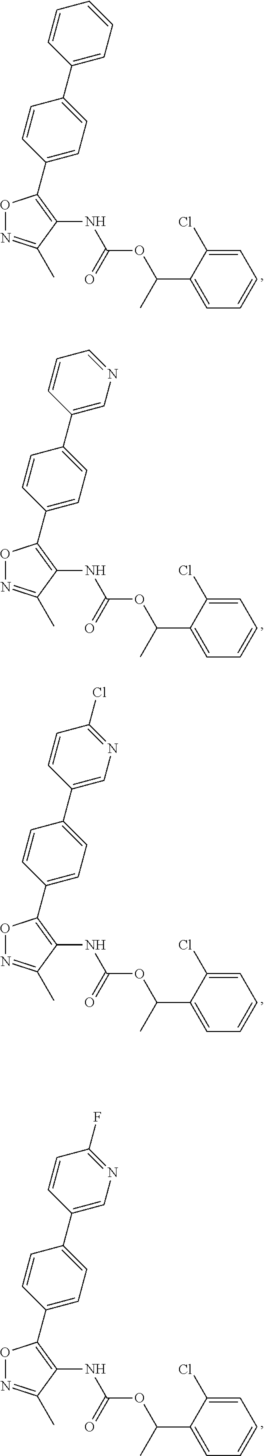 [SMILES: Cc1noc(-c2ccc(-c3ccccc3)cc2)c1NC(=O)OC(C)c1ccccc1Cl ; Cc1noc(-c2ccc(-c3cccnc3)cc2)c1NC(=O)OC(C)c1ccccc1Cl ; Cc1noc(-c2ccc(-c3ccc(Cl)nc3)cc2)c1NC(=O)OC(C)c1ccccc1Cl ; Cc1noc(-c2ccc(-c3ccc(F)nc3)cc2)c1NC(=O)OC(C)c1ccccc1Cl]

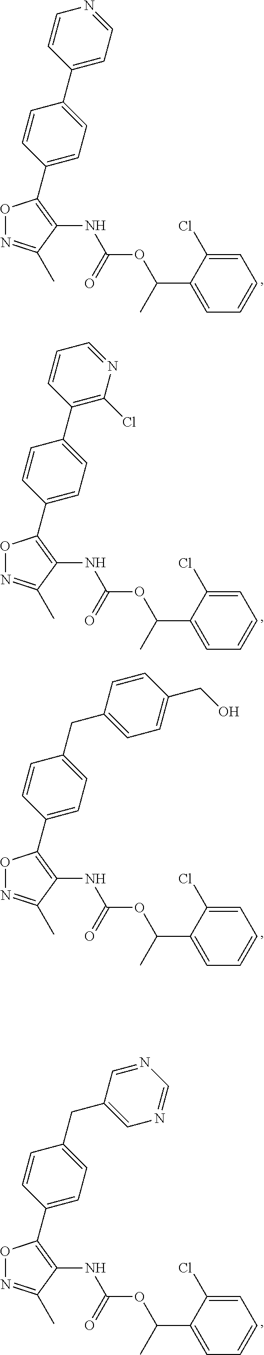

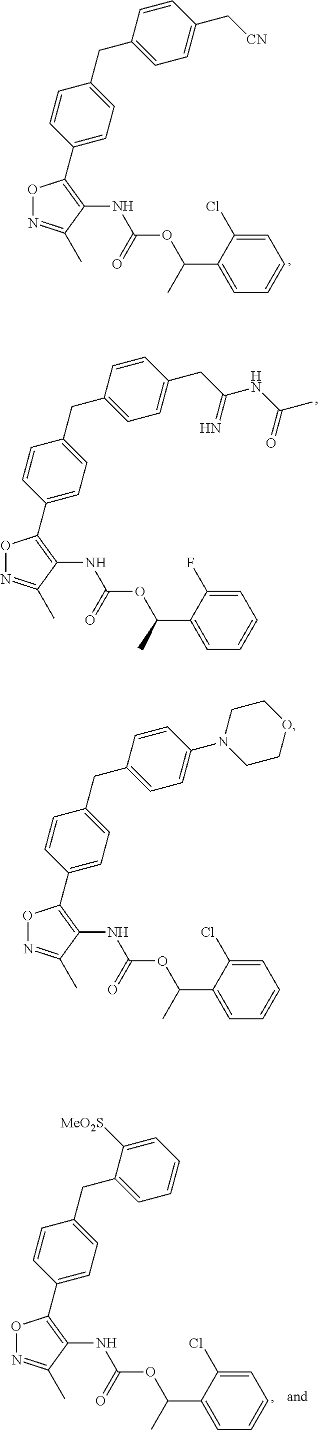 [SMILES: Cc1noc(-c2ccc(Cc3ccc(CC#N)cc3)cc2)c1NC(=O)OC(C)c1ccccc1Cl ; CC(=O)NC(=N)Cc1ccc(Cc2ccc(-c3onc(C)c3NC(=O)O[C@H](C)c3ccccc3F)cc2)cc1 ; Cc1noc(-c2ccc(Cc3ccc(N4CCOCC4)cc3)cc2)c1NC(=O)OC(C)c1ccccc1Cl ; Cc1noc(-c2ccc(Cc3ccccc3S(C)(=O)=O)cc2)c1NC(=O)OC(C)c1ccccc1Cl]

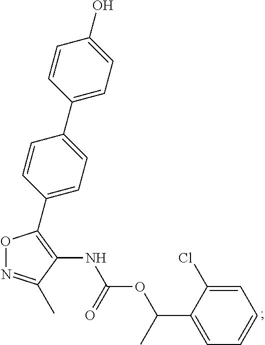 [SMILES: Cc1noc(-c2ccc(-c3ccc(O)cc3)cc2)c1NC(=O)OC(C)c1ccccc1Cl]

or a pharmaceutically acceptable salt, prodrug, active metabolite, or a pharmaceutically acceptable solvate thereof.

In some embodiments, the LPA receptor antagonist has the structure of Formula (VI) or a pharmaceutically acceptable salt thereof:

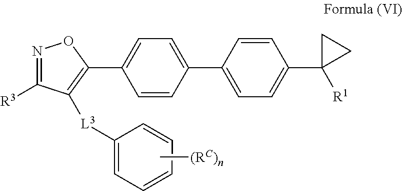

wherein,
R¹ is —CO₂R^{D}, —C(═O)NHSO₂R^{E}, —C(═O)N(R^{D})₂, or tetrazolyl;
   R^{D} is H or C₁-C₆alkyl;
   R^{E} is C₁-C₆alkyl, C₃-C₆cycloalkyl, or substituted or unsubstituted phenyl;
L³ is a substituted or unsubstituted C₃-C₆alkylene, a substituted or unsubstituted C₃-C₆fluoroalkylene, or a substituted or unsubstituted C₃-C₆heteroalkylene, where if L³ is substituted then L³ is substituted with 1, 2 or 3 le; each R¹³ is independently F, C₁-C₄alkyl, C₁-C₄fluoroalkyl, or —OH;
each R^{C} is independently halogen, —CN, —NO₂, —OH, C₁-C₄alkyl, C₁-C₄fluoroalkyl, C₁-C₄fluoroalkoxy, C₁-C₄alkoxy, or C₁-C₄heteroalkyl;
R³ is H or C₁-C₄alkyl;
n is 0, 1, or 2.

For any and all of the embodiments, substituents are selected from among from a subset of the listed alternatives. For example, in some embodiments, R¹ is —CO₂R^{D} or —C(═O)NHSO₂R^{E}. In some embodiments, R¹ is —CO₂R^{D}. In some embodiments, R¹ is —CO₂H. In some embodiments, R¹ is —C(═O)NHSO₂R^{E}. In some embodiments, R^{E} is C₁-C₆alkyl. In some embodiments, R^{E} is —CH₃ or —CH₂CH₃. In some embodiments, R^{E} is —CH₃. In some embodiments, R^{D} is H, —CH₃ or —CH₂CH₃. In some embodiments, R^{D} is —CH₂CH₃. In some embodiments, R^{D} is H.

In some embodiments, each R^{C} is independently halogen, —CN, —OH, —CH₃, —CH₂CH₃, —CH(CH₃)₂, —CF₃, —OCF₃, —OCH₃ or —OCH₂CH₃. In some embodiments, n is 0 or 1. In some embodiments, n is 0. In some embodiments, n is 1. In some embodiments, n is 2.

In some embodiments,

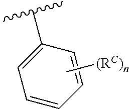

is phenyl, 2-fluorophenyl, 2,3-difluorophenyl, 2,4-difluorophenyl, 2,5-difluorophenyl, 2,6-difluorophenyl, 2-chlorophenyl, 2,6-dichlorophenyl, 2-bromophenyl, 3-bromophenyl, 2,4-dichlorophenyl, 2-hydroxyphenyl, 3-hydroxyphenyl, 4-hydroxyphenyl, 2-methoxyphenyl, 3-methoxyphenyl, 4-methoxyphenyl, 2-trifluoromethylphenyl, 3-trifluoromethylphenyl, 4-trifluoromethylphenyl, 2-fluoro-4-methoxyphenyl, 2-methylphenyl, 3-methylphenyl, 4-methylphenyl, 2-cyanophenyl, 3-cyanophenyl, or 4-cyanophenyl. In some embodiments,

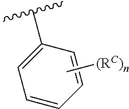

is phenyl, 2-fluorophenyl, 2-chlorophenyl, 2-trifluoromethylphenyl, or 2-methylphenyl. In some embodiments,

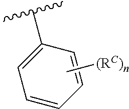

is phenyl, 2-fluorophenyl, or 2-chlorophenyl. In some embodiments,

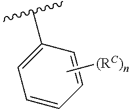

is phenyl.

In some embodiments, R³ is —H, —CH₃ or —CH₂CH₃. In some embodiments, R³ is —CH₃ or —CH₂CH₃. In some embodiments, R³ is —CH₃.

In some embodiments, R¹ is —CO₂R^{D}, or —C(═O)NHSO₂R^{E}; R^{D} is H or C₁-C₄alkyl; R^{E} is C₁-C₄alkyl; R³ is —H, —CH₃ or —CH₂CH₃.

In some embodiments, L³ is a substituted or unsubstituted C₃-C₄alkylene, a substituted or unsubstituted C₃-C₄fluoroalkylene, or a substituted or unsubstituted C₃-C₆heteroalkylene; where if L³ is substituted then L³ is substituted with 1, 2 or 3 R¹³; each R¹³ is independently F, —CH₃, —CH₂CH₃, —CF₃, or —OH.

In some embodiments, L³ is a substituted or unsubstituted butylene, a substituted or unsubstituted fluorobutylene, or a substituted or unsubstituted difluorobutylene; where if L³ is substituted then L³ is substituted with 1 or 2 R³.

In some embodiments, L³ is a substituted or unsubstituted C₃-C₆heteroalkylene; where if L³ is substituted then L³ is substituted with 1 or 2 R³.

In some embodiments, L³ is -(substituted or unsubstituted C₃-C₄alkylene)-O—, -(substituted or unsubstituted C₁-C₃alkylene)-O-(substituted or unsubstituted C₁-C₃alkylene)-, —O-(substituted or unsubstituted C₃-C₄alkylene)-, -(substituted or unsubstituted C₃-C₄alkylene)-S—, -(substituted or unsubstituted C₁-C₃alkylene)-S-(substituted or unsubstituted C₁-C₃alkylene)-, —S-(substituted or unsubstituted C₃-C₄alkylene)-, -(substituted or unsubstituted C₃-C₄alkylene)-NH—, -(substituted or unsubstituted C₁-C₃alkylene)-NH-(substituted or unsubstituted C₁-C₃alkylene)-, or —NH-(substituted or unsubstituted C₃-C₄alkylene)-.

In some embodiments, L³ is —NH-(substituted or unsubstituted C₃-C₄alkylene); where if L³ is substituted then L³ is substituted with R³.

In some embodiments, L³ is -(substituted or unsubstituted C₁-C₃alkylene)-O-(substituted or unsubstituted C₁-C₃alkylene)-, or -(substituted or unsubstituted C₁-C₃alkylene)-S-(substituted or unsubstituted C₁-C₃alkylene)-; where if L³ is substituted then L³ is substituted with R³.

In some embodiments, L³ is -(substituted or unsubstituted ethylene)-O-(substituted or unsubstituted methylene)-, or -(substituted or unsubstituted ethylene)-S-(substituted or unsubstituted methylene)-; where if L³ is substituted then L³ is substituted with R³.

In some embodiments, L³ is a substituted or unsubstituted C₃-C₄alkylene, a substituted or unsubstituted C₃-C₄fluoroalkylene, or a substituted or unsubstituted C₃-C₆heteroalkylene.

In some embodiments, L³ is a substituted or unsubstituted butylene, a substituted or unsubstituted fluorobutylene, or a substituted or unsubstituted difluorobutylene.

In some embodiments, L³ is a substituted or unsubstituted C₃-C₆heteroalkylene. In some embodiments, L³ is a substituted or unsubstituted C₃-C₄heteroalkylene.

In some embodiments, L³ is —NH-(substituted or unsubstituted C₃-C₄alkylene).

In some embodiments, L³ is -(substituted or unsubstituted C₁-C₃alkylene)-O-(substituted or unsubstituted C₁-C₃alkylene)-, or -(substituted or unsubstituted C₁-C₃alkylene)-S-(substituted or unsubstituted C₁-C₃alkylene)-. In some embodiments, L³ is -(substituted or unsubstituted ethylene)-O-(substituted or unsubstituted methylene)-, or -(substituted or unsubstituted ethylene)-S-(substituted or unsubstituted methylene)-.

In some embodiments, L³ is substituted with 1, 2 or 3 R¹³. In some embodiments, L³ is substituted with 1 or 2 R¹³. In some embodiments, L³ is substituted with R¹³. In some embodiments, L³ is unsubstituted. In some embodiments, if L³ is substituted then L³ is substituted with 1, 2 or 3 R¹³. In some embodiments, if L³ is substituted then L³ is substituted with 1 or 2 R¹³. In some embodiments, if L³ is substituted then L³ is substituted with R¹³. In some embodiments, L³ is unsubstituted. In some embodiments, each R¹³ is independently F, C₁-C₄alkyl, C₁-C₄fluoroalkyl, or —OH. In some embodiments, each R¹³ is independently F, C₁-C₄alkyl, or —OH. In some embodiments, each R¹³ is independently C₁-C₄alkyl, or —OH. In some embodiments, each R¹³ is independently F, —CH₃, —CH₂CH₃, —CF₃, or —OH. In some embodiments, each R¹³ is independently F, —CH₃, or —OH. In some embodiments, each R¹³ is independently —CH₃, or —OH. In some embodiments, R¹³ is F, —CH₃, —CH₂CH₃, —CF₃, or —OH. In some embodiments, R¹³ is F, —CH₃, —CH₂CH₃, or —OH. In some embodiments, R¹³ is —CH₃ or —OH. In some embodiments, R¹³ is C₁-C₄alkyl, or —OH.

In some embodiments, the LPA receptor antagonist has the structure of Formula (VII) or a pharmaceutically acceptable salt thereof:

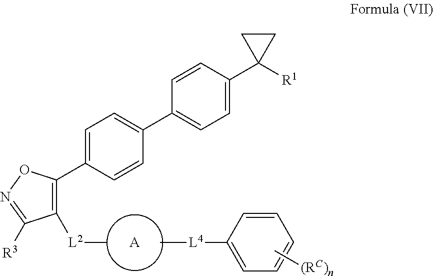

wherein,
R¹ is —CO₂R^{D}, —C(═O)NHSO₂R^{E}, —C(═O)N(R^{D})₂, —CN, or tetrazolyl;
   R^{D}is H or C₁-C₆ alkyl;
   R^{E} is C₁-C₆ alkyl or a substituted or unsubstituted phenyl;
L² is absent, —C(═O)—, —N(R^{D})—, substituted or unsubstituted C₁-C₄ alkylene, or substituted or unsubstituted C₁-C₄ heteroalkylene, where if L² is substituted, then L² is substituted with R¹², where R¹² is F, C₁-C₄alkyl, —OH, or —OR^{D};
ring A is a substituted or unsubstituted phenyl, or a substituted or unsubstituted monocyclic C₁-C₅heteroarylene, where if ring A is substituted, then ring A is substituted with 1 or 2 R¹⁴, each R¹⁴ is independently selected from halogen, —CN, —OH, C₁-C₄alkyl, C₁-C₄fluoroalkyl, C₁-C₄fluoroalkoxy, C₁-C₄alkoxy, and C₁-C₄heteroalkyl;
L⁴ is absent, or a substituted or unsubstituted C₁-C₄ alkylene, where if L⁴ is substituted then L⁴ is substituted with R¹³, where R¹³ is F, C₁-C₄alkyl, —OH, or —OR^{D};
R³ is H or C₁-C₄ alkyl;
each R^{C} is independently selected from halogen, —CN, —OH, C₁-C₄alkyl, C₁-C₄fluoroalkyl, C₁-C₄fluoroalkoxy, C₁-C₄alkoxy, and C₁-C₄heteroalkyl;
n is 0, 1 or 2.

For any and all of the embodiments, substituents are selected from among from a subset of the listed alternatives. For example, in some embodiments, R¹ is —CO₂R^{D} or —C(═O)NHSO₂R^{E}. In some embodiments, R¹ is —CO₂R^{D}. In some embodiments, R¹ is —CO₂H. In some embodiments, R¹ is —C(═O)NHSO₂R^{E}. In some embodiments, R^{E} is C₁-C₆ alkyl. In some embodiments, R^{E} is —CH₃ or —CH₂CH₃. In some embodiments, R^{D} is H, —CH₃ or —CH₂CH₃. In some embodiments, R^{D} is H.

In some embodiments, R³ is C₁-C₄alkyl. In some embodiments, R³ is H, —CH₃, or —CH₂CH₃. In some embodiments, R³ is —CH₃, or —CH₂CH₃. In some embodiments, R³ is —CH₃. In some embodiments, R³ is H.

In some embodiments, L² is absent, —C(═O)—, —N(R^{D})—, substituted or unsubstituted C₁-C₄ alkylene, or substituted or unsubstituted C₁-C₄heteroalkylene, where if L² is substituted, then L² is substituted with R¹². In some embodiments, L² is —N(R^{D})—, substituted or unsubstituted C₁-C₂ alkylene, or substituted or unsubstituted C₁-C₂ heteroalkylene, where if L² is substituted, then L² is substituted with R¹². In some embodiments, L² is —N(H)—, —N(CH₃)—, substituted or unsubstituted methylene, or substituted or unsubstituted ethylene, where if L² is substituted, then L² is substituted with R¹². In some embodiments, L² is —N(H)—. In some embodiments, L² is substituted or unsubstituted methylene, where if L² is substituted, then L² is substituted with R¹².

In some embodiments, L² is selected from a bond, C₁-C₄alkylene, —C(═O)—, —CH(OH)—, —CH(OR^{D})—, —CH₂CH(OH)—, —CH₂CH(OR^{D})—, —CH₂S—, —CH₂S(O)—, —CH₂S(O)₂—, —SCH₂—, —S(O)CH₂—, —S(O)₂CH₂—, —CH₂O—, —OCH₂—, —S(O)₂CH₂—, —N(H)—, —CH₂N(H)—, or —N(H)CH₂—.

In some embodiments, L² is absent, —C(═O)—, —NH—, —N(CH₃)—, —CH₂—, —CH₂CH₂—, —CH(CH₃)—, —CH₂CH(CH₃)—, —CH(CH₃)CH₂—, —CH(OH)—, —CH(OR^{D})—, —CH₂CH(OH)—, —CH₂CH(OR^{D})—, —CH(OH)CH₂—, —CH(OR^{D})CH₂—, —CH₂NH—, —CH(CH₃)NH—, —NHCH₂— or —NHCH(CH₃)—. In some embodiments, L² is —NH—, —N(CH₃)—, —CH₂—, —CH(CH₃)—, —CH(OH)—, —CH(OR^{D})—, —CH₂NH—, —CH(CH₃)NH—, —NHCH₂— or —NHCH(CH₃)—. In some embodiments, L² is —NH—, —N(CH₃)—, —CH₂NH—, —CH(CH₃)NH—, —NHCH₂— or —NHCH(CH₃)—. In some embodiments, L² is —NH—. In some embodiments, L² is —CH₂—, —CH(CH₃)—, —CH(OH)—, —CH(OR^{D})—, —CH₂NH—, —CH(CH₃)NH—, —NHCH₂— or —NHCH(CH₃)—. In some embodiments, L² is —CH₂—, —CH(CH₃)—, —CH(OH)—, or —CH(OR^{D}). In some embodiments, L² is —CH₂— or —CH(OH)—. In some embodiments, L² is —CH(OH)—. In some embodiments, L² is —CH₂—.

In some embodiments, R¹² is F, —CH₃, —CH₂CH₃, —OH, —OCH₃, or —OCH₂CH₃. In some embodiments, R¹² is —CH₃, or —OH.

In some embodiments, R¹ is —CO₂R^{D}, or —C(═O)NHSO₂R^{E}; L² is absent, —C(═O)—, —NH—, —N(CH₃)—, —CH₂—, —CH₂CH₂—, —CH(CH₃)—, —CH₂CH(CH₃)—, —CH(CH₃)CH₂—, —CH(OH)—, —CH(OR^{D})—, —CH₂CH(OH)—, —CH₂CH(OR^{D})—, —CH(OH)CH₂—, —CH(OR^{D})CH₂—, —CH₂NH—, —CH(CH₃)NH—, —NHCH₂— or —NHCH(CH₃)—; L⁴ is absent, —CH₂—, —CH(CH₃)—, —CH(OH)—, —CH₂CH₂—, —CH₂CH(CH₃)—, —CH(CH₃)CH₂—, —CH₂CH(OH)—, or —CH(OH)CH₂—; R³ is —H, —CH₃ or —CH₂CH₃.

In some embodiments, each R^{C} is halogen, —OH, —CH₃, —CH₂CH₃, —CF₃, —OCF₃, —OCH₃, —OCH₂CH₃, —CH₂OCH₃, —CH₂OCH₂CH₃, or —CH₂N(CH₃)₂. In some embodiments, each R^{C} is independently selected from halogen, —OH, —CH₃, —CH₂CH₃, —CF₃, —OCF₃, —OCH₃ and —OCH₂CH₃.

In some embodiments, ring A is a substituted or unsubstituted phenyl, or a substituted or unsubstituted monocyclic C₁-C₅heteroarylene, where if ring A is substituted, then ring A is substituted with 1 or 2 R¹⁴.

In some embodiments, ring A is a substituted or unsubstituted phenyl, or a substituted or unsubstituted monocyclic C₁-C₅heteroarylene containing 1-4 N atoms, 0 or 1 O atoms and 0 or 1 S atoms, where if ring A is substituted, then ring A is substituted with 1 or 2 R¹⁴.

In some embodiments, ring A is a substituted or unsubstituted monocyclic C₁-C₅heteroarylene containing 1-4 N atoms, 0 or 1 O atoms and 0 or 1 S atoms, where if ring A is substituted, then ring A is substituted with 1 or 2 R¹⁴.

In some embodiments, ring A is a substituted or unsubstituted 5-membered monocyclic C₁-C₄heteroarylene containing 1-4 N atoms, 0 or 1 O atoms and 0 or 1 S atoms, where if ring A is substituted, then ring A is substituted with 1 or 2 R¹⁴.

In some embodiments, ring A is a substituted or unsubstituted 6-membered monocyclic C₃-C₅heteroarylene containing 1-3 N atoms, where if ring A is substituted, then ring A is substituted with 1 or 2 R¹⁴.

In some embodiments, ring A is a substituted or unsubstituted monocyclic ring wherein the groups -L²- and -L⁴- are in a 1,2-relationship on ring A (i.e. an ortho relationship). In some embodiments, ring A is a substituted or unsubstituted monocyclic ring wherein the groups -L²- and -L⁴- are in a 1,3-relationship on ring A (i.e. a meta relationship). In some embodiments, ring A is a substituted or unsubstituted monocyclic ring wherein the groups -L²- and -L⁴- are in a 1,4-relationship on ring A (i.e. a para relationship).

In some embodiments, ring A is unsubstituted or monosubstituted with R¹⁴. In some embodiments, ring A is unsubstituted. In some embodiments, ring A is monosubstituted with R¹⁴.

In some embodiments, L⁴ is absent, or a substituted or unsubstituted methylene, or substituted or unsubstituted ethylene, where if L⁴ is substituted, then L⁴ is substituted with R¹³. In some embodiments, L⁴ is absent. In some embodiments, L⁴ is a substituted or unsubstituted methylene, where if L⁴ is substituted, then L⁴ is substituted with R¹³. In some embodiments, L⁴ is a substituted or unsubstituted ethylene, where if L⁴ is substituted, then L⁴ is substituted with R¹³

In some embodiments, R¹³ is F, —CH₃, —CH₂CH₃, —OH, —OCH₃, or —OCH₂CH₃. In some embodiments, R¹³ is —CH₃.

In some embodiments, L⁴ is absent, —CH₂—, or —CH(CH₃)—.

In some embodiments, L² is —NH—, —CH₂—, —CH₂CH₂—, —CH(CH₃)—, —CH₂CH(CH₃)—, —CH(CH₃)CH₂—, —CH(OH)—, —CH₂CH(OH)—, —CH(OH)CH₂—, —CH₂NH—, —CH(CH₃)NH—, —NHCH₂— or —NHCH(CH₃)—; ring A is a substituted or unsubstituted phenyl, or a substituted or unsubstituted monocyclic C₁-C₅heteroarylene containing 1-4 N atoms, 0 or 1 O atoms and 0 or 1 S atoms, where if ring A is substituted, then ring A is substituted with 1 or 2 R¹⁴; L⁴ is absent, —CH₂—, or —CH(CH₃)—; R³ is —CH₃.

In some embodiments, ring A is a substituted or unsubstituted phenyl, where if ring A is substituted, then ring A is substituted with 1 or 2 R¹⁴.

In some embodiments, ring A is a substituted or unsubstituted monocyclic C₁-C₅heteroarylene containing 1-4 N atoms, 0 or 1 O atoms and 0 or 1 S atoms, where if ring A is substituted, then ring A is substituted with 1 or 2 R¹⁴.

In some embodiments, ring A is a substituted or unsubstituted 5-membered monocyclic C₁-C₄heteroarylene containing 1-4 N atoms, 0 or 1 O atoms and 0 or 1 S atoms, where if ring A is substituted, then ring A is substituted with 1 or 2 R¹⁴.

In some embodiments, ring A is a substituted or unsubstituted furanyl, a substituted or unsubstituted thienyl, substituted or unsubstituted pyrrolyl, substituted or unsubstituted oxazolyl, substituted or unsubstituted thiazolyl, substituted or unsubstituted imidazolyl, substituted or unsubstituted pyrazolyl, substituted or unsubstituted triazolyl, substituted or unsubstituted tetrazolyl, substituted or unsubstituted isoxazolyl, substituted or unsubstituted isothiazolyl, substituted or unsubstituted oxadiazolyl, or substituted or unsubstituted thiadiazolyl, where if ring A is substituted, then ring A is substituted with 1 or 2 R¹⁴

In some embodiments, each R¹⁴ is independently selected from halogen, —CN, —OH, —CH₃, —CH₂CH₃, —CF₃, —OCF₃, —OCH₃ and —OCH₂CH₃. In some embodiments, each R¹⁴ is halogen, —CN, —OH, —CH₃, —CH₂CH₃, —CF₃, —OCF₃, —OCH₃ or —OCH₂CH₃. In some embodiments, each R¹⁴ is independently selected from halogen, —OH, and —CH₃. In some embodiments, R¹⁴ is halogen, —OH, or —CH₃. In some embodiments, each R¹⁴ is independently selected from halogen and —CH₃.

In some embodiments, ring A is

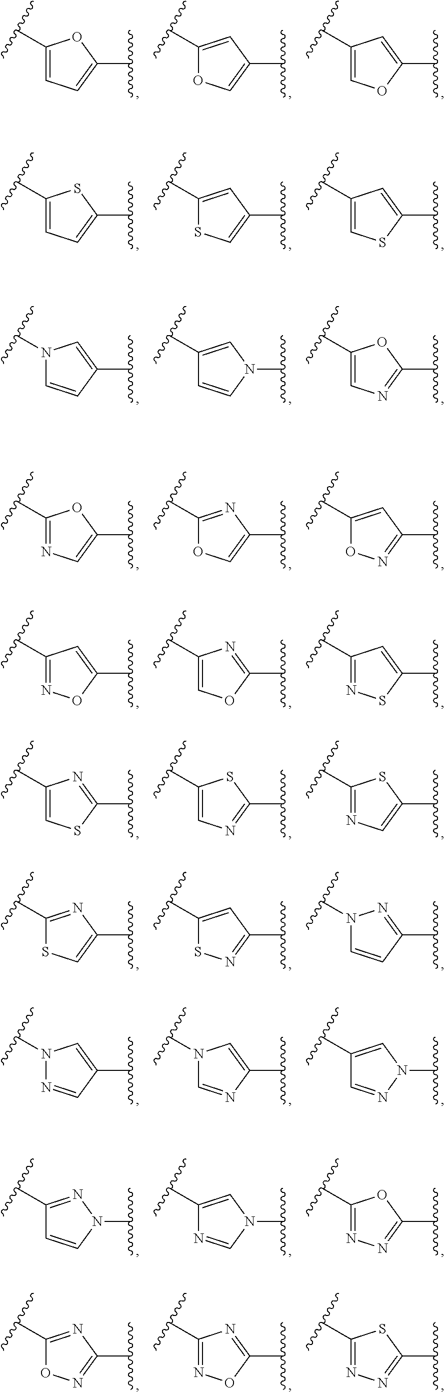

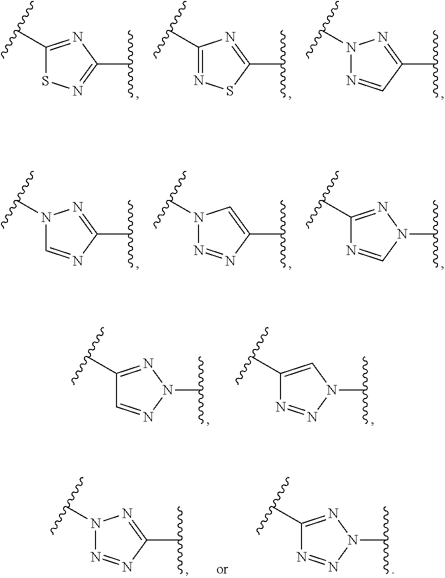

In some embodiments, ring A is a substituted or unsubstituted 6-membered monocyclic C₃-C₅heteroarylene containing 1-3 N atoms, where if ring A is substituted, then ring A is substituted with 1 or 2 R¹⁴.

In some embodiments, ring A is a substituted or unsubstituted pyridinylene, a substituted or unsubstituted pyridazinylene, a substituted or unsubstituted pyrimidinylene, a substituted or unsubstituted pyrazinylene, or a substituted or unsubstituted triazinylene, where if ring A is substituted, then ring A is substituted with 1 or 2 R¹⁴.

In some embodiments, ring A is a substituted or unsubstituted pyridinylene, where if ring A is substituted, then ring A is substituted with 1 or 2 R¹⁴.

In some embodiments, ring A is

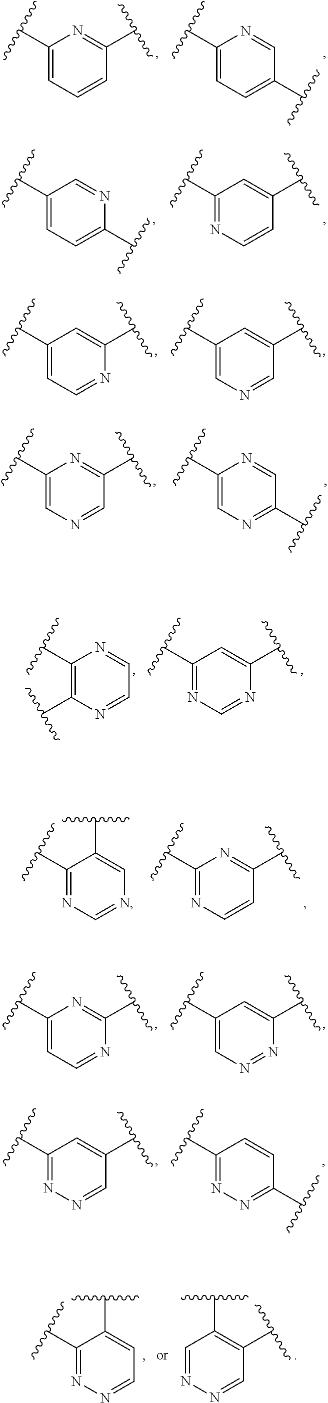

In some embodiments, R¹ is —CO₂R^{D}, or —C(═O)NHSO₂R^{E}; R^{D} is H or C₁-C₄ alkyl; R^{E} is C₁-C₄ alkyl; L² is —CH₂—, —CH(CH₃)—, or —CH(OH)—; ring A is a substituted or unsubstituted 5-membered monocyclic C₁-C₄heteroarylene containing 1-4 N atoms, 0 or 1 O atoms and 0 or 1 S atoms, where if ring A is substituted, then ring A is substituted with R¹⁴; L⁴ is —CH₂— or —CH(CH₃)—; p is 0 or 1.

In some embodiments, R¹ is —CO₂R^{D}, or —C(═O)NHSO₂R^{E}; R^{D} is H or C₁-C₄ alkyl; R^{E} is C₁-C₄ alkyl; L² is —CH₂—, —CH(CH₃)—, or —CH(OH)—; ring A is a substituted or unsubstituted 5-membered monocyclic C₁-C₄heteroarylene containing 1-4 N atoms and 0 or 1 O atoms, where if ring A is substituted, then ring A is substituted with R¹⁴, R¹⁴ is halogen, —CN, —OH, —CH₃, —CH₂CH₃, —CF₃, —OCF₃, —OCH₃ or —OCH₂CH₃; L⁴ is —CH₂— or —CH(CH₃)—; n is 0 or 1.

In some embodiments, R¹ is —CO₂R^{D}, or —C(═O)NHSO₂R^{E}; R^{D} is H or C₁-C₄alkyl; R^{E} is C₁-C₄alkyl; L² is —NH—, —CH₂—, —CH(CH₃)—, —CH(OH)—, —NHCH₂— or —NHCH(CH₃)—; ring A is a substituted or unsubstituted 6-membered monocyclic C₃-C₅heteroarylene containing 1-3 N atoms, where if ring A is substituted, then ring A is substituted with R¹⁴; L⁴ is absent, —CH₂—, or —CH(CH₃)—; p is 0 or 1.

In some embodiments, R¹ is —CO₂R^{D}, or —C(═O)NHSO₂R^{E}; R^{D} is H or C₁-C₄alkyl; R^{E} is C₁-C₄alkyl; L² is —NH—, —CH₂—, —CH(CH₃)—, —CH(OH)—, —NHCH₂— or —NHCH(CH₃)—; ring A is a substituted or unsubstituted pyridinylene, where if ring A is substituted, then ring A is substituted with R¹⁴, R¹⁴ is halogen, —CN, —OH, —CH₃, —CH₂CH₃, —CF₃, —OCF₃, —OCH₃ or —OCH₂CH₃; L⁴ is absent, —CH₂—, or —CH(CH₃)—; n is 0 or 1.

In some embodiments, n is 0, 1 or 2. In some embodiments, n is 0 or 1. In some embodiments, n is 0. In some embodiments, n is 1.

In some embodiments,

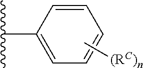

is phenyl, 2-fluorophenyl, 2,3-difluorophenyl, 2,4-difluorophenyl, 2,5-difluorophenyl, 2,6-difluorophenyl, 2-chlorophenyl, 2,6-dichlorophenyl, 2-bromophenyl, 3-bromophenyl, 2,4-dichlorophenyl, 2-hydroxyphenyl, 3-hydroxyphenyl, 4-hydroxyphenyl, 2-methoxyphenyl, 3-methoxyphenyl, 4-methoxyphenyl, 2-trifluoromethylphenyl, 3-trifluoromethylphenyl, 4-trifluoromethylphenyl, 2-fluoro-4-methoxyphenyl, 2-methylphenyl, 3-methylphenyl, 4-methylphenyl, 2-cyanophenyl, 3-cyanophenyl, or 4-cyanophenyl.

Any combination of the groups described above for the various variables is contemplated herein. Throughout the specification, groups and substituents thereof are chosen by one skilled in the field to provide stable moieties and compounds.

In some embodiments, the LPA1 receptor antagonist is selected from compounds (or a pharmaceutically acceptable salt, prodrug, active metabolite, or a pharmaceutically acceptable solvate thereof) described in U.S. Pat. Nos. 6,964,975; 7,288,558 and U.S. Application Publication No. 2006/0194850, each of which is herein incorporated by reference.

In some embodiments, the LPA1 receptor antagonist is 3-[[[4-[4-[[[1-(2-chlorophenyl)ethoxy]carbonyl]amino]-3-methyl-5-isoxazolyl]phenyl]methyl]thio]-propanoic acid, or a pharmaceutically acceptable salt, prodrug, active metabolite, or a pharmaceutically acceptable solvate thereof.

In some embodiments, the LPA1 receptor antagonists contemplated for use herein (including pharmaceutically acceptable salts, solvates, and prodrugs thereof) are antagonists of LPA1 and optionally at least one of the LPA receptors selected from LPA₂, LPA₃, LPA₄, LPA₅ and LPA₆. In one embodiment, the LPA1 receptor antagonists are antagonists LPA₁ and/or LPA₃. In some embodiments, the LPA1 compounds are antagonists of LPA₁ and/or LPA₂.

In one aspect, LPA1 receptor antagonists contemplated for use in ay of the embodiments disclosed herein are selective LPA1 receptor antagonists. “Selectivity” for one LPA receptor versus other LPA receptors means that the compound has an IC₅₀ (Ca Flux assay) for the indicated LPA receptor that is at least 10-fold less than the IC₅₀ for other LPA receptors. In some embodiments, selectivity for one LPA receptor versus other LPA receptor means that the compound has an IC₅₀ for the indicated LPA receptor that is at least 10-fold, at least 20-fold, at least 40-fold, at least 50-fold, at least 100-fold, at least 200-fold, at least 500-fold, or at least 1000-fold, less than the IC₅₀ for other LPA receptors. For example, a selective LPA₁ receptor antagonist has an IC₅₀ that is at least 10-fold, at least 20-fold, at least 40-fold, at least 50-fold, at least 100-fold, at least 200-fold, at least 500-fold, or at least 1000-fold, less than the IC₅₀ for other LPA receptors (e.g. LPA₂, LPA₃).

In some embodiments, pharmaceutically acceptable salts are obtained by reacting an LPA1 receptor antagonist compound with acids. Pharmaceutically acceptable salts are also obtained by reacting an LPA1 receptor antagonist compound with a base. In one aspect LPA1 receptor antagonists described herein are used as pharmaceutically acceptable salts. The type of pharmaceutical acceptable salts, include, but are not limited to: (1) acid addition salts, formed by reacting the free base form of the compound with a pharmaceutically acceptable: inorganic acid to form a salt such as, for example, a hydrochloric acid salt, a hydrobromic acid salt, a sulfuric acid salt, a phosphoric acid salt, a metaphosphoric acid salt, and the like; or with an organic acid to form a salt, such as, for example, an acetic acid salt, a propionic acid salt, a hexanoic acid salt, a cyclopentanepropionic acid salt, a glycolic acid salt, a pyruvic acid salt, a lactic acid salt, a malonic acid salt, a succinic acid salt, a malic acid salt, a maleic acid salt, a fumaric acid salt, a trifluoroacetic acid salt, a tartaric acid salt, a citric acid salt, a benzoic acid salt, a 3-(4-hydroxybenzoyl)benzoic acid salt, a cinnamic acid salt, a mandelic acid salt, a methanesulfonic acid salt, an ethanesulfonic acid salt, a 1,2-ethanedisulfonic acid salt, a 2-hydroxyethanesulfonic acid salt, a benzenesulfonic acid salt, a toluenesulfonic acid salt, a 2-naphthalenesulfonic acid salt, a 4-methylbicyclo-[2.2.2]oct-2-ene-1-carboxylic acid salt, a glucoheptonic acid salt, a 4,4′-methylenebis-(3-hydroxy-2-ene-1-carboxylic acid) salt, a 3-phenylpropionic acid salt, a trimethylacetic acid salt, a tertiary butylacetic acid salt, a lauryl sulfuric acid salt, a gluconic acid salt, a glutamic acid salt, a hydroxynaphthoic acid salt, a salicylic acid salt, a stearic acid salt, a muconic acid salt, a butyric acid salt, a phenylacetic acid salt, a phenylbutyric acid salt, a valproic acid salt, and the like; (2) salts formed when an acidic proton present in the parent compound is replaced by a metal ion, e.g., an alkali metal ion (e.g. a lithium salt, a sodium salt, potassium salt), an alkaline earth ion (e.g. a magnesium salt, or a calcium salt), or an aluminum ion (e.g. an aluminum salt). In some cases, LPA1 receptor antagonist compounds described herein are reacted with an organic base to form a salt, such as, but not limited to, an ethanolamine salt, a diethanolamine salt, a triethanolamine salt, a tromethamine salt, a N-methylglucamine salt, a dicyclohexylamine salt, a tris(hydroxymethyl)methylamine salt. In other cases, LPA receptor antagonist compounds described herein form salts with amino acids such as, but not limited to, an arginine salt, a lysine salt, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like. In some embodiments, LPA1 receptor antagonist compounds are prepared and utilized as a sodium salt.

In some embodiments, the LPA1 receptor antagonist compounds described herein possess one or more stereocenters and each center exists independently in either the R or S configuration. The compounds presented herein include all diastereomeric, enantiomeric, and epimeric forms as well as the appropriate mixtures thereof.

In some embodiments, LPA1 antagonists presented herein are used as a single enantiomer. In some embodiments, LPA1 antagonists presented herein are used as a single enantiomer that is optically pure (i.e. substantially free of the other isomer). In some embodiments, LPA1 antagonists presented herein are used as a single enantiomer of any optical purity. In some embodiments, the opposite enantiomer of a LPA1 antagonist presented herein is used (of any optical purity). In some embodiments, LPA1 antagonists presented herein are used as a racemic mixture.

Any combination of the groups described above for the various variables is contemplated herein. Throughout the specification, groups and substituents thereof can be chosen by one skilled in the field to provide stable moieties and compounds.

In certain embodiments, the compounds presented herein possess one or more stereocenters and each center independently exists in either the R or S configuration. The compounds presented herein include all diastereomeric, enantiomeric, and epimeric forms as well as the appropriate mixtures thereof. Stereoisomers are obtained, if desired, by methods such as, stereoselective synthesis and/or the separation of stereoisomers by chiral chromatographic columns.

The methods and formulations described herein include the use of N-oxides (if appropriate), crystalline forms (also known as polymorphs), or pharmaceutically acceptable salts of compounds having the structures presented herein, as well as active metabolites of these compounds having the same type of activity. In some situations, compounds may exist as tautomers. All tautomers are included within the scope of the compounds presented herein. In specific embodiments, the compounds described herein exist in solvated forms with pharmaceutically acceptable solvents such as water, ethanol, and the like. In other embodiments, the compounds described herein exist in unsolvated form.

### Certain Terminology

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Unless otherwise indicated, conventional methods of mass spectroscopy, NMR, HPLC, protein chemistry, biochemistry, recombinant DNA techniques and pharmacology are employed. In this application, the use of “or” or “and” means “and/or” unless stated otherwise. Furthermore, use of the term “including” as well as other forms, such as “include”, “includes,” and “included,” is not limiting. The section headings used herein are for organizational purposes only and are not to be construed as limiting the subject matter described.

An “alkyl” group refers to an aliphatic hydrocarbon group. The alkyl group may be a saturated alkyl group or an unsaturated alkyl group. The alkyl moiety, whether saturated or unsaturated, may be branched or straight chain. Typical alkyl groups include, but are in no way limited to, methyl, ethyl, propyl, isopropyl, butyl, isobutyl, sec-butyl, tertiary butyl, pentyl, neopentyl, hexyl, allyl, but-2-enyl, but-3-enyl, and the like. In one aspect the alkyl is selected from the group consisting of methyl, ethyl, propyl, iso-propyl, n-butyl, iso-butyl, sec-butyl, and t-butyl.

The term “alkylene” refers to a divalent alkyl radical. Any of the above mentioned monovalent alkyl groups may be an alkylene by abstraction of a second hydrogen atom from the alkyl. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

An “alkoxy” group refers to a (alkyl)O— group, where alkyl is as defined herein.

“Aryl” refers to phenyl or naphthalenyl. In one aspect, an aryl is a phenyl. Depending on the structure, an aryl group can be a monoradical or a diradical (i.e., an arylene group). Exemplary arylenes include, but are not limited to, phenyl-1,2-ene, phenyl-1,3-ene, and phenyl-1,4-ene.

The term “cycloalkyl” refers to a monocyclic or polycyclic aliphatic, non-aromatic radical, wherein each of the atoms forming the ring (i.e. skeletal atoms) is a carbon atom. Cycloalkyls may be saturated, or partially unsaturated. In some embodiments, cycloalkyl groups are selected from among cyclopropyl, cyclobutyl, cyclopentyl, cyclopentenyl, cyclohexyl, cyclohexenyl, cycloheptyl, and cyclooctyl. Depending on the structure, a cycloalkyl group can be a monoradical or a diradical (i.e., an cycloalkylene group, such as, but not limited to, cyclopropan-1,1-diyl, cyclobutan-1,1-diyl, cyclopentan-1,1-diyl, cyclohexan-1,1-diyl, cyclohexan-1,4-diyl, cycloheptan-1,1-diyl, and the like).

The term “halo” or, alternatively, “halogen” or “halide” means fluoro, chloro, bromo or iodo.

The term “haloalkyl” refers to an alkyl group in which one or more hydrogen atoms are replaced by one or more halide atoms.

The term “haloalkylene” refers to an alkylene group in which one or more hydrogen atoms are replaced by one or more halide atoms.

The term “fluoroalkyl” refers to an alkyl in which one or more hydrogen atoms are replaced by a fluorine atom.

The term “fluoroalkylene” refers to an alkylene in which one or more hydrogen atoms are replaced by a fluorine atom.

The term “heteroalkyl” refers to an alkyl group in which one or more skeletal atoms of the alkyl are selected from an atom other than carbon, e.g., oxygen, nitrogen (e.g. NH or Nalkyl), sulfur, or combinations thereof.

The term “heteroalkylene” refers to an alkylene group in which one or more skeletal atoms of the alkyl are selected from an atom other than carbon, e.g., oxygen, nitrogen, sulfur, or combinations thereof. Exemplary heteroalkylenes include, but are not limited to, —OCH₂—, —OCH(CH₃)—, —OC(CH₃)₂—, —OCH₂CH₂—, —CH₂O—, —CH(CH₃)O—, —C(CH₃)₂O—, —CH₂CH₂O—, —CH₂OCH₂—, —CH₂OCH₂CH₂—, —CH₂CH₂OCH₂—, —SCH₂—, —SCH(CH₃)—, —SC(CH₃)₂—, —SCH₂CH₂—, —CH₂S—, —CH(CH₃)S—, —C(CH₃)₂S—, —CH₂CH₂S—, —CH₂SCH₂—, —CH₂SCH₂CH₂—, —CH₂CH₂SCH₂—, —SO₂CH₂—, —SO₂CH(CH₃)—, —SO₂C(CH₃)₂—, —SO₂CH₂CH₂—, —CH₂SO₂—, —CH(CH₃)SO₂—, —C(CH₃)₂SO₂—, —CH₂CH₂SO₂—, —CH₂SO₂CH₂—, —CH₂SO₂CH₂CH₂—, —CH₂CH₂SO₂CH₂—, —NHCH₂—, —NHCH(CH₃)—, —NHC(CH₃)₂—, —NHCH₂CH₂—, —CH₂NH—, —CH(CH₃)NH—, —C(CH₃)₂NH—, —CH₂CH₂NH—, —CH₂CH₂CH₂NH—, —NHCH₂CH₂CH₂—, —CH₂NHCH₂—, —CH₂NHCH₂CH₂—, —CH₂CH₂NHCH₂—, and the like.

The term “heteroaryl” refers to an aromatic ring that includes one or more ring heteroatoms selected from nitrogen, oxygen and sulfur. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. In one aspect, a heteroaryl contains 0-3 N atoms. In another aspect, a heteroaryl contains 1-3 N atoms. In another aspect, a heteroaryl contains 0-3 N atoms, 0-1 O atoms, and 0-1 S atoms. In another aspect, a heteroaryl is a monocyclic or bicyclic heteroaryl. In one aspect, heteroaryl is a C₁-C₉heteroaryl. In one aspect, monocyclic heteroaryl is a C₁-C₅heteroaryl. In one aspect, monocyclic heteroaryl is a 5-membered or 6-membered heteroaryl. In one aspect, bicyclic heteroaryl is a C₆-C₉heteroaryl. Depending on the structure, a heteroaryl group can be a monoradical or a diradical (i.e., a heteroarylene group).

A “heterocycloalkyl” refers to a cycloalkyl group that includes at least one heteroatom selected from nitrogen, oxygen and sulfur. In some embodiments, the heterocycloalkyl is selected from oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, and indolinyl. The term heteroalicyclic also includes all ring forms of the carbohydrates, including but not limited to the monosaccharides, the disaccharides and the oligosaccharides. In one aspect, a heterocycloalkyl is a C₂-C₁₀heterocycloalkyl. In another aspect, a heterocycloalkyl is a C₄-C₁₀heterocycloalkyl. In one aspect, a heterocycloalkyl contains 0-2 N atoms. In another aspect, a heterocycloalkyl contains 0-2 N atoms, 0-2 O atoms or 0-1 S atoms.

The term “membered ring” is meant to denote the number of skeletal atoms that constitute the ring. Thus, for example, cyclohexyl, pyridinyl, pyranyl and thiopyranyl are 6-membered rings and cyclopentyl, pyrrolyl, furanyl, and thienyl are 5-membered rings.

The term “moiety” refers to a specific segment or functional group of a molecule. Chemical moieties are often recognized chemical entities embedded in or appended to a molecule.

The term “optionally substituted” or “substituted” means that the referenced group may be substituted with one or more additional group(s) individually and independently selected from halogen, —CN, —NH₂, —OH, —NH(CH₃), —N(CH₃)₂, —CO₂H, —CO₂alkyl, —C(═O)NH₂, —C(═O)NHalkyl, —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, —S-alkyl, or —S(═O)₂alkyl. In some embodiments, an optional substituent is selected from halogen, —CN, —NH₂, —OH, —NH(CH₃), —N(CH₃)₂, —CH₃, —CH₂CH₃, —CF₃, —OCH₃, and —OCF₃. In some embodiments, substituted groups are substituted with one or two of the preceding groups. In some embodiments, substituted groups are substituted with one of the preceding groups.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

The term “antagonist,” as used herein, refers to a molecule such as a compound, which diminishes, inhibits, or prevents the action of another molecule or the activity of a receptor site. Antagonists include, but are not limited to, competitive antagonists, non-competitive antagonists, uncompetitive antagonists, partial agonists and inverse agonists.

The term “LPA-dependent”, as used herein, refers to conditions or disorders that would not occur, or would not occur to the same extent, in the absence of LPA.

The term “LPA-mediated”, as used herein, refers to refers to conditions or disorders that might occur in the absence of LPA but can occur in the presence of LPA.

“Ophthalmic formulation” and “ocular formulation” are interchangeable.

The terms “effective amount” or “therapeutically effective amount,” as used herein, refer to a sufficient amount of an agent or a compound (e.g. an LPA receptor antagonist described herein) being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of an LPA receptor antagonist that is required to provide a clinically significant decrease in disease symptoms. An appropriate “effective” amount in any individual case may be determined using techniques, such as a dose escalation study.

The term “subject” or “individual” or “patient” encompasses mammals and non-mammals. In one embodiment, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The terms “prevent,” “preventing,” or “prevention” and other grammatical equivalents used herein include inhibiting (arresting or stopping) the development of a disorder and/or inhibiting (arresting or stopping) the further progression of a disorder. These terms are meant to include prophylaxis. For prophylactic benefit, the compositions are administered to an individual suspected of having a particular disorder, at risk of developing a disorder or to an individual reporting one or more symptoms of a disorder or at risk of reocurrence of a disease.

The terms “ocular fibrosis” or “fibrosing disorder,” as used herein, refers to conditions that are associated with the abnormal accumulation of cells, fibronectin and/or collagen, and/or increased fibroblast recruitment or proliferation and include but are not limited to fibrosis and/or aberrant wound healing of individual organs or tissues such as ocular tissues.

The term “cancer,” or “proliferative disorder” or “proliferative condition” as used herein refers to an abnormal growth of cells which tend to proliferate in an uncontrolled way and, in some cases, to metastasize (spread). The type of cancer includes, but is not limited to, epithelial cancers, melanoma, or lymphoma at any stage of the disease with or without metastases.

### Formulations

Disclosed herein, in certain embodiments, is a formulation of a LPA1 antagonist wherein the formulation is suitable for local or systemic administration.

In some embodiments, the formulation is an oral formulation. In some embodiments, the formulation is a parenteral (e.g., intravenous, subcutaneous, intramuscular) formulation. In some embodiments, the formulation is a topical formulation for administration to the eye.

Pharmaceutical formulations disclosed herein are formulated in any suitable manner. Any suitable technique, carrier, and/or excipient is contemplated for use with the LPA1 antagonist. For a summary of pharmaceutical formulations described herein see Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Eighth Ed. (Lippincott Williams & Wilkins 2004), Muller, R. H. et al. Advanced Drug Delivery Reviews 59 (2007) 522-530, which are herein incorporated by reference for such disclosures.

### Systemic Administration

In some embodiments, an LPA receptor antagonist is delivered to a target site in the eye of a mammal through systemic administration. In certain embodiments, a pharmaceutical formulations comprising an LPA receptor antagonist is optionally administered by multiple administration routes, including, but not limited to, oral and parenteral (e.g., intravenous, subcutaneous, intramuscular) routes of administration. Parenteral injections optionally involve bolus injections or continuous infusions. The pharmaceutical formulations, include, but are not limited to, solutions, suspensions, aqueous liquid dispersions, self-emulsifying dispersions, solid solutions, liposomal dispersions, solid dosage forms, powders, immediate release formulation, controlled release formulations, fast melt formulations, tablets, capsules, pills, delayed release formulations, extended release formulations, pulsatile release formulations, multiparticulate formulations, and mixed immediate and controlled release formulations. Generally speaking, one will desire to administer an amount of an LPA1 antagonist is that is effective to achieve a plasma level commensurate with the concentrations found to be effective in vivo for a period of time effective to elicit a therapeutic effect.

### Oral Formulations

In certain embodiments, an LPA1 antagonist is formulated in a manner that is suitable for oral administration to a mammal.

For oral administration, an LPA1 antagonist is formulated by combining the active compound with pharmaceutically acceptable carriers or excipients. Such carriers enable the LPA1 antagonist to be formulated as tablets, powders, pills, dragees, capsules, liquids, gels, syrups, elixirs, slurries, suspensions and the like, for oral ingestion by a mammal.

The pharmaceutical compositions will include at least one pharmaceutically acceptable carrier, diluent or excipient and an LPA1 antagonist as an active ingredient in free-acid or free-base form, or in a pharmaceutically acceptable salt form.

The oral solid dosage formulations described herein include particles of an LPA1 antagonist in crystalline form, amorphous form, semi-crystalline form, semi-amorphous form, or mixtures thereof.

In one embodiment, the pharmaceutical compositions described herein are formulated into any suitable dosage form, including but not limited to, aqueous oral dispersions, solid oral dosage forms, fast melt formulations, lyophilized formulations, tablets, capsules, extended release formulations, IV formulations.

In one embodiment, an LPA1 antagonist is formulated into an immediate release form that provides for once-a-day administration.

In some embodiments, the solid dosage forms described herein are in the form of a tablet, (including an immediate release tablet, an extended release tablet, a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, e.g., capsules made from animal-derived gelatin or plant-derived HPMC, or “sprinkle capsules”), solid dispersion, multiparticulate dosage forms, pellets, or granules.

In one embodiment, a capsule is prepared. In some embodiments, the formulations (non-aqueous suspensions and solutions) are placed in a soft gelatin capsule. In other embodiments, the formulations are placed in standard gelatin capsules or non-gelatin capsules such as capsules comprising HPMC.

Liquid formulation dosage forms for oral administration include, but not limited to, pharmaceutically acceptable aqueous oral dispersions, emulsions, solutions, and syrups. See, e.g., Singh et al., Encyclopedia of Pharmaceutical Technology, 2^{nd} Ed., pp. 754-757 (2002).

### Parenteral Formulations

Disclosed herein, in certain embodiments, is a formulation of a LPA1 antagonist wherein the formulation is suitable for parenteral administration.

In some embodiments, the LPA1 antagonist is formulated for intramuscular, subcutaneous, or intravenous injection. In some embodiments, the LPA1 antagonist is formulated as a suspension, solution or emulsion.

In some embodiments, the parenteral formulation comprises a pharmaceutically-acceptable excipient. In some embodiments, the parenteral formulation comprises a carrier, suspending agent, thickening agent, stabilizing agent, wetting agent, emulsifying agent, dispersing agent, preservative, antioxidant, buffer, an isotonizing agent, or a combination thereof.

Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants.

Suitable lipophilic solvents or vehicles include fatty oils such as sesame oil, or synthetic fatty acid esters, such as ethyl oleate or triglycerides, or liposomes.

For intravenous injections, the LPA1 antagonist is optionally formulated in aqueous solutions, preferably in physiologically compatible buffers such as Hank's solution, Ringer's solution, or physiological saline buffer. For other parenteral injections, appropriate formulations include aqueous or nonaqueous solutions, preferably with physiologically compatible buffers or excipients.

Formulations for injection are optionally presented in unit dosage form (e.g., in ampoules or vials) or in multi dose containers. In some embodiments, a parenteral formulations is stored in powder form or in a freeze-dried (lyophilized) condition requiring only the addition of the sterile liquid carrier, for example, saline or sterile pyrogen-free water, immediately prior to use.

In some embodiments, a parenteral formulation disclosed herein is formulated as a depot preparation. Such long acting formulations may be administered by implantation (for example subcutaneously or intramuscularly) or by intramuscular injection. Thus, for example, the compounds may be formulated with suitable polymeric or hydrophobic materials (e.g., as an emulsion) or ion exchange resins, or as sparingly soluble derivatives, for example, as a sparingly soluble salt.

### Ophthalmic Formulations

Ophthalmic formulations described herein deliver LPA receptor antagonists to the ophthalmic tissues of a mammal. In certain instances, localized ocular administration of an LPA receptor antagonist reduces or eliminates side-effects associated with systemic administration of LPA receptor antagonists. The formulations described herein are administered as eye drops, ointment or gel, and are suitable for delivery of LPA receptor antagonists to the eyes of a mammal. In some instances, the ophthalmic formulation is an ocular insert for insertion into the cul-de-sac of the conjunctiva between the sclera of the eyeball and the lid that dispenses drug to the eye. Ophthalmic formulations described herein include but are not limited to solutions, suspensions, gels, ointments, drops, or inserts.

### Solutions

Described herein is an ophthalmic formulation comprising an LPA receptor antagonist wherein the ophthalmic formulation is in the form of a solution. In certain instances, delivery of an LPA receptor antagonist is achieved by administration of an ocular solution formulation as drops to the eye of a mammal. In certain instances, the solution is administered as an eye wash to the eyes of a mammal.

In certain instances, the solution comprises an LPA receptor antagonist or a salt thereof dissolved in sterile water and/or 0.9% sodium chloride solution. Small quantities of an alcohol or glycerin are optionally used to solubilize the LPA receptor antagonist compound. The quantities of alcohol and/or glycerin are kept as low as possible to minimize irritation to the eye upon administration. Any irritation to ocular tissues results in excessive tearing and washing away of the administered formulation.

In some instances, the solutions comprise a pH modifying agent to solubilize the LPA receptor antagonist. In certain instances, a pH-modifying agent maintains a solution pH of 5-8 and solubilizes the LPA receptor antagonist (e.g., an acid salt of an LPA receptor antagonist). In certain instances, the solution further comprises a preservative and/or a stabilizer. In certain instances, sterile solutions are obtained in the absence of a preservative and/or a stabilizer using filtration systems (e.g., 0.2μ filtration systems) and/or heat treatment. The preservative and/or stablizer is present in an amount from about 0.001% to about 5% of the total weight of the formulation. In some embodiments, the ocular solutions described herein further comprise tonicity agents. Examples of tonicity agents that are compatible with the formulations described herein include and are not limited to sodium borate, boric acid, sodium chloride, potassium chloride, mannitol, dextrose, glycerin, propylene glycol or mixtures thereof. The solutions are designed for isotonicity with physiological fluids (e.g., osmolality of the solution compositions is about 300 mOsm).

### Suspensions and Emulsions

Described herein, in certain embodiments, is an ophthalmic formulation comprising an LPA-receptor antagonist wherein the ophthalmic formulation is in the form of an emulsion or a suspension. The ophthalmic formulation that is in the form of an emulsion or a suspension is suitable for administration of LPA receptor antagonists to the eye of a mammal. The ophthalmic formulation that is in the form of an emulsion a suspension is administered topically as eye drops in a mammal. The formulations further comprise pH-modifying agents, preservatives and/or stabilizers. In certain instances, sterile formulations are obtained in the absence of a preservative and/or a stabilizer using filtration systems (e.g., 0.2μ filtration systems) and/or heat treatment. In certain embodiments, the suspensions or emulsions comprise a surfactant to enhance solubility of an LPA receptor antagonist. In certain embodiments, the surfactant concentration is kept as low as possible to minimize foaming that might interfere with proper administration. In certain instances, the liquid phase (e.g., a cosolvent) of a suspension or emulsion has a density similar to the density of the suspensoid. In certain instances, the liquid phase is a cosolvent that partially dissolves or does not dissolve the LPA receptor antagonist, thus minimizing particle size growth resulting from the dissolved compound crystallizing out onto crystals present in the suspenoid.

In some embodiments the suspensions or emulsions are aqueous suspensions or emulsions. The aqueous suspensions optionally comprise suspending agents, such as for example, sodium carboxymethylcellulose, methylcellulose, hydroxypropyl-methylcellulose, sodium alginate, polyvinylpyrrolidone, gum tragacanth, gum acacia or the like or mixtures thereof.

In some embodiments, the suspensions or emulsions are oil-in-water suspensions or emulsions. The oily phase is a vegetable oil, (e.g., olive oil, castor oil, soy oil, sesame oil, coconut oil) or a mineral oil (e.g., liquid paraffin). Such suspensions or emulsions optionally contain a thickening agent, for example beeswax, hard paraffin or cetyl alcohol.

The oil-in-water suspensions or emulsions optionally comprise emulsifying agents such as naturally-occurring gums, for example, gum acacia or gum tragacanth, naturally-occurring phosphatides, for example, soya bean lecithin, and esters or partial esters derived from fatty acids and hexitol anhydrides, for example, sorbitan mono-oleate, and condensation products of the said partial esters with ethylene oxide, for example, polyoxyethylene sorbitan mono-oleate, or the like.

In certain embodiments, an ophthalmic formulation described herein is isotonic with physiological fluids (e.g., osmolality of about 290 mOsm). In certain instances, the suspensions, emulsions or colloidal dispersions comprise tonicity agents (e.g., sodium chloride, potassium chloride or the like) that render the formulation isotonic with physiological fluids. In certain instances, an ophthalmic formulation described herein is a hypotonic formulation. Hypotonic formulations allow for absorption of the LPA receptor antagonist from the lacrimal sac.

### Ointments

Disclosed herein, in certain embodiments, is a topical formulation for administration to an eye wherein the topical formulation for ocular administration is in the form of an ointment. In certain instances, ointments are semisolid (e.g., soft solid or thick liquid) formulations that include an LPA receptor antagonist compound dispersed in an oil-in-water emulsion or a water-in-oil emulsion. In some embodiments, the hydrophobic component of an ointment is derived from an animal (e.g., lanolin, cod liver oil, and ambergris), plant (e.g., safflower oil, castor oil, coconut oil, cottonseed oil, menhaden oil, palm kernel oil, palm oil, peanut oil, soybean oil, rapeseed oil, linseed oil, rice bran oil, pine oil, sesame oil, or sunflower seed oil), or petroleum (e.g., mineral oil, or petroleum jelly). In certain instances, ointments are semisolid preparations that soften or melt at body temperature (including the temperature of an eye and/or a tissue related thereto). In certain instances, ointments re-hydrate a tissue and are thus useful for ophthalmic disorders characterized by loss of moisture or dryness in the eye.

### Gels

Often there is difficulty in instilling eye drops in the eye. Ophthalmic drops also cause unpleasant side effects of tearing, eyelid crusting and/or vision blurring. The tearing causes difficulty in applying an appropriate amount of an LPA receptor antagonist to the eye due to rapid loss of drug through the lacrimal drainage system. Ophthalmic gels provide for good ocular retention, while avoiding burst release of an LPA-receptor antagonist. In some instances, an ophthalmic formulation is designed for controlled release of the LPA receptor antagonist in order to provide increased delivery efficiency, and maximum therapeutic effect.

Described herein, in certain embodiments, is an ophthalmic formulation comprising an LPA receptor antagonist wherein the ophthalmic formulation is in the form of a liquid that gels upon administration to the eye. The formulation is a liquid at room temperature and comprises thermosensitive and/or pH sensitive gelling polymers in an aqueous base. Application of such a liquid formulation to the eye causes rapid gellation of the polymer (i.e. a transition from liquid phase to solid phase) thereby preventing tearing and washing away of the LPA receptor antagonist.

Ophthalmic formulations comprising thermosensitive gelling polymers are liquids at room temperature and gel at body temperature. Thermally-sensitive gelling polymers include alkyl cellulose, hydroxyalkyl cellulose, cellulosic ethers, Pluronic polymers,Tetronic polymers or the like, or mixtures thereof. Ophthalmic formulations described comprising pH-sensitive gelling polymers gel upon mixing with aqueous tear fluid. pH sensitive gelling polymers include acidic and crosslinked acidic polymers such as those containing carboxyl groups (e.g., carboxy vinyl polymers such as polyacrylates, crosslinked polyacrylate acid, methacrylic acid, ethacrylic acid, β-methylacrylic acid, cis-α-methylcrotonic acid, trans-α-methylcrotonic acid, α-butylcrotonic acid, α-phenylacrylic acid, α-benzylacrylic acid, α-cyclohexylacrylic acid, and the like or mixtures thereof.

Injection into the Eye

In some embodiments, a formulation of an LPA receptor antagonist for administration to a tissue of the eye is administered or delivered via injection to a target site of the eye, where it releases an LPA receptor antagonist over a defined period of time. In certain embodiments, a formulation of an LPA receptor antagonist is administered by intravitreal injection. In some embodiments, a formulation of an LPA receptor antagonist is administered by subtenon injection. In certain embodiments, a formulation of an LPA receptor antagonist is administered by retrobulbar injection. In some embodiments, the formulation for injection of an LPA receptor antagonist is a solution formulation. In certain embodiments, the formulation for injection of an LPA receptor antagonist is a suspension formulation.

### Ocular Inserts

In some embodiments, a formulation of an LPA receptor antagonist for administration to an eye is administered or delivered via a device that can be inserted between an eye and eyelid or in the conjunctival sac, where it releases the LPA receptor antagonist. In certain embodiments, an LPA receptor antagonist is released into the lacrimal fluid that bathes the surface of the cornea, or directly to the cornea itself, with which the solid device is generally in intimate contact. In certain embodiments, a suitable device for administration to an eye is used with an LPA receptor antagonist (e.g., an eyegate applicator).

In some embodiments, a depot preparation for insertion in the eye is formulated by forming microencapsulated matrices (also known as microencapsule matrices) of an LPA receptor antagonist in biodegradable polymers. In some embodiments, a depot preparation is formulated by entrapping an LPA receptor antagonist in liposomes or microemulsions.

In some instances an ophthalmic formulation described herein comprises nano-particles of an LPA receptor antagonist. In some instances ophthalmic formulations described herein comprise crystalline particles. In some embodiments, ophthalmic formulations described herein comprise amorphous particles. In some embodiments, a topical formulation for administration to an eye disclosed herein is administered or delivered to the posterior segments of an eye (e.g., to the retina, choroid, vitreous and optic nerve). In some embodiments, an ophthalmic formulation described herein is applied to the surface of the eye or in the lacrimal sac or under the eyelid.

### Ocular Implants

In some embodiments, an LPA receptor antagonist for administration to an eye is administered or delivered via an injectable or implantable depot preparation. As used herein, a depot preparation is a controlled-release formulation enclosed within a device that is implanted in an eye or a tissue related thereto (e.g., the sclera) (for example subcutaneously, intramuscularly, intravitreally, or within the subconjunctiva). The ratio of LPA receptor antagonist to controlled-release matrix and the nature of the matrix employed control the rate of drug release.

### Excipients

Described herein, in certain embodiments, is an ophthalmic formulation for delivery to the eyes of a mammal that comprises one or more pH adjusting agents. Examples of pH adjusting agents or buffering agents, include, but are not limited to acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Described herein, in certain embodiments, is an ophthalmic formulation for delivery to the eyes of a mammal that comprises one or more tonicity agents. A topical formulation for administration to an eye has an ophthalmically acceptable tonicity. In certain instances, lacrimal fluid has an isotonicity value equivalent to that of a 0.9% sodium chloride solution. In certain instances, an isotonicity value from about 0.6% to about 1.8% sodium chloride equivalency is suitable for topical administration to an eye. In certain instances, a topical formulation for administration to an eye disclosed herein has an osmolarity from about 200 to about 600 mOsm/L. In some embodiments, a topical formulation for administration to an eye disclosed herein is hypotonic and thus requires the addition of any suitable to attain the proper tonicity range. Tonicity agents are used to adjust the composition of the formulation to the desired isotonic range. Tonicity agents include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate. Other exemplary tonicity agents include mannitol, dextrose,

Described herein, in certain embodiments, is an ophthalmic formulation for delivery to the eyes of a mammal that comprises one or more preservatives to inhibit microbial activity. Suitable preservatives include benzoic acid, boric acid, p-hydroxybenzoates, alcohols, mercury-containing substances such as merfen and thiomersal; stabilized chlorine dioxide; and quaternary ammonium compounds such as benzalkonium chloride, cetyltrimethylammonium bromide and cetylpyridinium chloride. In certain embodiments, the formulations described herein optionally include one or more stabilizers (e.g., antioxidants) to enhance chemical stability where required. Suitable antioxidants include, by way of example only, ascorbic acid, methionine, sodium thiosulfate and sodium metabisulfite. In one embodiment, antioxidants are selected from metal chelating agents, thiol containing compounds and other general stabilizing agents.

Described herein, in certain embodiments, is an ophthalmic formulation for delivery to the eyes of a mammal that comprises one or more surfactants. “Surfactants” are wetting agents that lower the surface tension of a liquid. Examples of surfactants for ophthalmic formulations include and are not limited to oils derived from natural sources, such as, corn oil, olive oil, cotton seed oil and sunflower seed oil; sorbitan esters, such as Sorbitan trioleate available under the trade name Span 85, Sorbitan mono-oleate available under the trade name Span 80, Sorbitan monolaurate available under the trade name Span 20, Polyoxyethylene (20) sorbitan monolaurate available under the trade name Tween 20, Polyoxyethylene (20) sorbitan mono-oleate available under the trade name Tween 80; lecithins derived from natural sources such as those available under the trade name Epikuron particularly Epikuron 200. Oleyl polyoxyethylene (2) ether available under the trade name Brij 92, Stearyl polyoxyethylene (2) available under the trade name Brij 72, Lauryl polyoxyethylene (4) ether available under the trade name Brij 30, Oleyl polyoxyethylene (2) ether available under the trade name Genapol 0-020, Block copolymers of oxyethylene and oxypropylene available under the trade name Synperonic, Oleic acid, Synthetic lecithin, Diethylene glycol dioleate, Tetrahydrofurfuryl oleate, Ethyl oleate, Isopropyl myristate, Glyceryl trioleate, Glyceryl monolaurate, Glyceryl mono-oleate, Glyceryl monostearate, Glyceryl monoricinoleate, Cetyl alcohol, Stearyl alcohol, Polyethylene glycol 400, and Cetyl pyridinium chloride.

In one embodiment, the aqueous suspensions or emulsions described herein remain in a homogenous state, as defined in The USP Pharmacists' Pharmacopeia (2005 edition, chapter 905), for at least 4 hours. In one embodiment, an aqueous suspension is re-suspended into a homogenous suspension by physical agitation lasting less than 1 minute (e.g., by shaking a eye-drop dispenser). In still another embodiment, no agitation is necessary to maintain a homogeneous aqueous dispersion.

In some embodiments, a topical formulation for administration to an eye comprises an ophthalmically acceptable viscosity enhancer (e.g., a thermo-sensitive or pH sensitive gelling polymer). In certain instances, a viscosity enhancer increases the time a formulation disclosed herein remains in an eye. In certain instances, increasing the time a formulation disclosed herein remains in the eye allows for greater drug absorption and effect. Non-limiting examples of mucoadhesive polymers include carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, a topical formulation for administration to an eye disclosed herein further comprises a solubilizing agent, for example, a glucan sulfate and/or a cyclodextrin. Glucan sulfates which can be used include, but are not limited to, dextran sulfate, cyclodextrin sulfate and β-1,3-glucan sulfate, both natural and derivatives thereof. Cyclodextrin derivatives that are used as a solubilizing agent include, but are not limited to, α-cyclodextrin, β-cyclodextrin, γ-cyclodextrin, hydroxyethyl β-cyclodextrin, hydroxypropyl γ-cyclodextrin, hydroxypropyl β-cyclodextrin, sulfated β-cyclodextrin, sulfated α-cyclodextrin, sulfobutyl ether β-cyclodextrin. The formulations described herein comprise from about 0.5 to 20% cyclodextrins. In some embodiments, the formulations described herein comprise from about 1 to about 10% cyclodextrins.

In some embodiments, the solution, emulsion or suspension formulations also include inert diluents commonly used in the art, such as water or other solvents, solubilizing agents, and/or emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In some embodiments, the ophthalmic formulations described herein are stable (e.g., with respect to pH, active ingredient) over a period of any of at least about 1 day, at least about 2 days, at least about 3 days, at least about 4 days, at least about 5 days, at least about 6 days, at least about 1 week, at least about 2 weeks, at least about 4 weeks, at least about 6 weeks, at least about 8 weeks, at least about 4 months, at least about 5 months, or at least about 6 months. In other embodiments, the formulations described herein are stable over a period of at least about 1 week to about 1 month, or at least about 1 month to about 6 months.

In certain embodiments, the ophthalmic formulations described herein are designed for minimal ophthalmic toxicity, irritation and/or allergic challenge to ocular tissues and include, for example, low amounts of excipients such as surfactants, preservatives and/or cosolvents.

### Methods of Dosing and Treatment Regimens

In one embodiment, LPA1 antagonists are used in the preparation of medicaments for the treatment of LPA-dependent or LPA-mediated diseases or conditions. In addition, a method for treating any of the diseases or conditions described herein in a subject in need of such treatment, involves administration of pharmaceutical compositions that include at least one LPA1 antagonist or a pharmaceutically acceptable salt, active metabolite, prodrug, or solvate thereof, in therapeutically effective amounts to said subject.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial.

In prophylactic applications, compositions containing the compounds described herein are administered to a patient susceptible to or otherwise at risk of a particular disease, disorder or condition.

In certain embodiments, the dose of drug being administered may be temporarily reduced or temporarily suspended for a certain length of time (i.e., a “drug holiday”).

For systemic modes of administration, generally speaking, one will desire to administer an amount of an LPA1 antagonist is that is effective to achieve a plasma level commensurate with the concentrations found to be effective in vivo for a period of time effective to elicit a therapeutic effect. Doses employed for adult human treatment are typically in the range of 0.01 mg-5000 mg per day or from about 1 mg to about 1000 mg per day. In one embodiment, the desired dose is conveniently presented in a single dose or in divided doses.

In some embodiments, formulations provide a therapeutically effective amount of an LPA1 antagonist, or a pharmaceutically acceptable salt thereof, enabling, for example, once a week, twice a week, three times a week, four times a week, five times a week, once every other day, once-a-day, twice-a-day (b.i.d.), or three times a day (t.i.d.) administration if desired. In one embodiment, the formulation provides a therapeutically effective amount of an LPA1 antagonist, or a pharmaceutically acceptable salt thereof, enabling once-a-day administration.

Also disclosed herein, are ophthalmic formulations of an LPA receptor antagonist compound wherein the ophthalmic formulation is administered for prophylactic and/or therapeutic treatments. In certain instances, amounts effective for this use will depend on the severity and course of the disease, disorder or condition, previous therapy, the mammal's health status and response to the drugs, and the judgment of the treating physician. In some embodiments, the dose of an LPA receptor antagonist is about 0.001% by weight to about 10% by weight of the ophthalmic formulation. In some embodiments, the dose of an LPA receptor antagonist is about 0.001% by weight to about 5% by weight of the ophthalmic formulation.

In some embodiments, where an LPA-dependent or LPA-mediated disease or condition does not improve, an ophthalmic formulation disclosed herein is administered chronically (i.e., for an extended period of time, including throughout the duration of the mammal's life). In some embodiments, where an LPA-dependent or LPA-mediated disease or condition does improve, an ophthalmic formulation disclosed herein is given continuously; alternatively, the dose of active agent being administered is temporarily reduced or temporarily suspended for a certain length of time (i.e., a “drug holiday”). In some embodiments, a drug holiday lasts between 2 days and 1 year, including all integers in between. In some embodiments, the dose reduction during a drug holiday is from about 10% to about 100%, including all integers in between.

In some embodiments, where an LPA-dependent or LPA-mediated disease or condition does improve, an ophthalmic formulation disclosed herein is administered as a maintenance dose. In some embodiments, where an LPA-dependent or LPA-mediated disease or condition does improve, an ophthalmic formulation disclosed herein is administered with reduced frequency or at a reduced dose.

In some embodiments, where an individual is suspected of having an LPA-dependent or LPA-mediated disease or condition, an ophthalmic formulation disclosed herein is administered as a prophylactic dose prior to onset of disease symptoms. In some embodiments, a prophylactic dose is a reduced dose compared to a therapeutic dose.

Useful ophthalmic formulations for administration to an eye can be aqueous or biphasic solutions, suspensions or solution/suspensions, or gels, which can be presented in the form of eye drops. A desired dosage can be administered via a set number of drops into an eye. For example, for a drop volume of 25 μl, administration of 1-6 drops will deliver 25-150 μl of a topical formulation for administration to an eye disclosed herein. Ophthalmic formulations described herein contain from about 0.01% to about 50%, more about 0.1% to about 20%, about 0.2% to about 10%, or from about 0.5% to about 5%, weight/volume of the formulation of an LPA receptor antagonist.

### Patient Selection

In any of the aforementioned aspects involving the prevention or treatment of LPA-mediated diseases or conditions of the eye are further embodiments comprising identifying patients by screening for LPA receptor gene SNPs. Patients can be further selected based on increased LPA receptor expression in the tissue of interest. LPA receptor expression are determined by methods including, but not limited to, northern blotting, western blotting, quantitative PCR (qPCR), flow cytometry, autoradiography (using a small molecule radioligand or PET ligand). In some embodiments, patients are selected based on the concentration of serum or tissue LPA measured by mass spectrometry. In some embodiments, patients are selected based on a combination of the above markers (increased LPA concentrations and increased LPA receptor expression).

### Combination Therapy

In one aspect, pharmaceutical compositions and methods disclosed herein include an additional therapeutic agent. In one aspect, the additional therapeutic agent is a therapeutic agent other than an LPA1 antagonist.

In one aspect, the pharmaceutical compositions disclosed herein that include an LPA1 antagonist are co-administered with (either separately or in the same formulation) a therapeutic agent selected from: antibiotics (e.g., polymyxin B sulfate/bacitracin zinc, polymyxin B/neomycin/gramicidin, polymyxin B/trimethoprim, polymyxin B/bacitracin, fluoroquinolones (e.g., ciprofloxacin, moxifloxacin, ofloxacin, gatifloxacin, levofloxacin), aminoglycosides (e.g. tobramycin, azithromycin, gentamicin, erythromycin, bacitracin); anti-Fungal Agents (e.g., amphotericin B, intraconazole, fluconazole, voriconazole); steroid anti-inflammatory agents (e.g., fluorometholone acetate, prednisolone acetate, loteprednol etabonate, prednisolone sodium phosphate, prednisolone sodium, rimexolone, fluorometholone acetate); non-steroidal anti-inflammatory agents (e.g., nepafenac, ketorolac tromethamine, bromfenac, diclofenac sodium, ketorolac tromethamine, ketotifen fumarate); antihistamines (e.g., emedastine difumarate, olopatadine hydrochloride, epinastine HCl, azelastine hydrochloride, ketotifen fumarate); antivirals (e.g., acyclovir, vidarabine, trifluridine); alpha agonists (e.g., apraclonidine, brimonidine, bimatoprost); beta blockers (e.g., betaxolol hydrochloride, levobunolol hydrochloride, carteolol hydrochloride, metipranolol, timolol maleate, timolol hemihydrate); carbonic anhydrase inhibitors (e.g., brinzolamide, dorzolamide, acetazolamide); miotics (e.g., acetylcholine chloride, echothiophate); prostaglandins (e.g., travoprost, bimatoprost, latanoprost); anti-angiogenesis agents (e.g., pegaptanib sodium, ranibizumab, verteporfin); loteprednol etabonate, mast cell stabilizers (e.g., lodoxamide tromethamine, nedocromil sodium, cromolyn sodium, pemirolast potassium), cyclosporine, and leukotriene modulators (e.g. 5-LO inhibitors, FLAP inhibitor compounds, leukotriene receptor antagonist (e.g. CysLT₁ receptor antagonists)).

In some embodiments, the pharmaceutical compositions disclosed herein comprising an LPA1 antagonist are co-administered with (either separately or in the same formulation) an antibiotic. Antibiotics include, but are not limited to polymyxin B sulfate/bacitracin zinc, polymyxin B/neomycin/gramicidin, polymyxin B/trimethoprim, polymyxin B/bacitracin, fluoroquinolones (e.g., ciprofloxacin, moxifloxacin, ofloxacin, gatifloxacin, levofloxacin), aminoglycosides (e.g. tobramycin, azithromycin, gentamicin, erythromycin, bacitracin).

In some embodiments, the pharmaceutical compositions disclosed herein comprising an LPA1 antagonist are co-administered with (either separately or in the same formulation) an anti-fungal agent. Anti-fungal agents include, but are not limited to amphotericin B, intraconazole, fluconazole, and voriconazole.

In some embodiments, the pharmaceutical compositions disclosed herein comprising an LPA1 antagonist are co-administered with (either separately or in the same formulation) a steroid anti-inflammatory agent. Steroid anti-inflammatory agents include but are not limited to, betamethasone, prednisone, alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, halcinonide, halometasone, hydrocortisone/cortisol, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone/prednisolone, rimexolone, tixocortol, triamcinolone, and ulobetasol.

In some embodiments, the pharmaceutical compositions disclosed herein comprising an LPA1 antagonist are co-administered with (either separately or in the same formulation) a non-steroidal anti-inflammatory agent (NSAID). NSAIDs include, but are not limited to, nepafenac, ketorolac, bromfenac, diclofenac, ketorolac, ketotifen.

In some embodiments, the pharmaceutical compositions disclosed herein comprising an LPA1 antagonist are co-administered with (either separately or in the same formulation) an antihistamine. In some embodiments, antihistamines include, but are not limited to, amelexanox, astemizole, azatadine, azelastine, acrivastine, brompheniramine, cetirizine, levocetirizine, efletirizine, chlorpheniramine, clemastine, cyclizine, carebastine, cyproheptadine, carbinoxamine, descarboethoxyloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, olopatadine, picumast, pyrilamine, promethazine, terfenadine, tripelennamine, temelastine, trimeprazine, and triprolidine. In some embodiments, antihistamines include, but are not limited to, emedastine, olopatadine, epinastine, azelastine, ketotifen.

In some embodiments, the pharmaceutical compositions disclosed herein comprising an LPA1 antagonist are co-administered with (either separately or in the same formulation) an antiviral agent. Antiviral agents include, but are not limited to, acyclovir, vidarabine, trifluridine.

In some embodiments, the pharmaceutical compositions disclosed herein comprising an LPA1 antagonist are co-administered with (either separately or in the same formulation) and alpha agonist. Alpha agonists include, but are not limited to, apraclonidine, brimonidine, bimatoprost.

In some embodiments, the pharmaceutical compositions disclosed herein comprising an LPA1 antagonist are co-administered with (either separately or in the same formulation) a beta blocker. Beta blockers include, but are not limited to, betaxolol, levobunolol, carteolol, metipranolol, timolol.

In some embodiments, the pharmaceutical compositions disclosed herein comprising an LPA1 antagonist are co-administered with (either separately or in the same formulation) a carbonic anhydrase inhibitor. Carbonic anhydrase inhibitors include, but are not limited to, brinzolamide, dorzolamide, acetazolamide.

In some embodiments, the pharmaceutical compositions disclosed herein comprising an LPA1 antagonist are co-administered with (either separately or in the same formulation) a miotic. Miotics include, but are not limited to, acetylcholine chloride, echothiophate.

In some embodiments, the pharmaceutical compositions disclosed herein comprising an LPA1 antagonist are co-administered with (either separately or in the same formulation) a prostaglandin. Prostaglandins include, but are not limited to, travoprost, bimatoprost, latanoprost.

In some embodiments, the pharmaceutical compositions disclosed herein comprising an LPA1 antagonist are co-administered with (either separately or in the same formulation) an anti-angiogenesis agent. Anti-angiogenesis agents include, but are not limited to, pegaptanib sodium, ranibizumab, verteporfin and bevacizumab.

In some embodiments, the pharmaceutical compositions disclosed herein comprising an LPA1 antagonist are co-administered with (either separately or in the same formulation) loteprednol etabonate.

In some embodiments, the pharmaceutical compositions disclosed herein comprising an LPA1 antagonist are co-administered with (either separately or in the same formulation) a mast cell stabilizer. Mast cell stabilizers include, but are not limited to, lodoxamide tromethamine, nedocromil sodium, cromolyn sodium, pemirolast potassium.

In some embodiments, the pharmaceutical compositions disclosed herein comprising an LPA1 antagonist are co-administered with (either separately or in the same formulation) cyclosporine.

In some embodiments, the pharmaceutical compositions disclosed herein comprising an LPA1 antagonist are co-administered with (either separately or in the same formulation) a leukotriene modulator. Leukotriene modulators include, but are not limited to 5-lipoxygenase (5-LO) inhibitors inhibitors, 5-lipoxygenase activating protein (FLAP) inhibitor compounds and leukotriene receptor antagonist (e.g. CysLT₁ receptor antagonists).

In some embodiments, the LPA1 antagonist and the additional therapeutic agent are in the same pharmaceutical composition. In some embodiments, the LPA1 antagonist and the additional therapeutic agent are in separate pharmaceutical compositions. In some embodiments, the LPA1 antagonist and the additional therapeutic agent are administered at the same time. In some embodiments, the LPA1 antagonist and the additional therapeutic agent are administered at different times.

### EXAMPLES

These examples are provided for illustrative purposes only and not to limit the scope of the claims provided herein.

### Example 1

### Ocular Emulsion Formulation (Preservative Free)

| Ingredient | Concentration (wt %) |
|---|---|
| LPA1 antagonist | 2% |
| Monobasic sodium phosphate | 0.13% |
| Dibasic sodium phosphate | 0.01% |
| Sodium Chloride | 0.4% (Osmolality 282 mOsm) |
| hydroxypropylcellulose | 0.5% |
| Sterile water | q.s. |
| Olive oil | 5% |
| HCl/NaOH | pH 5 |

LPA1 antagonist is ball milled for approximately 4 h. A hydroxypropylmethyulcelluslose vehicle is prepared by mixing 2% aqueous hydroxypropylmethylcellulose, sodium chloride, dibasic sodium phosphate, disodium edetate, sodium chloride, and water together and the pH is adjusted to 7.4 by the addition of 1N HCl. Olive oil and LPA1 antagonist are added to a portion of the vehicle and the mixture is mixed well to furnish the desired suspension. The suspension is sterilized by heat treatment and packaged in sterile containers.

### Example 2

### Ocular Suspension Formulation with Preservative

| Ingredient | Concentration (wt %) |
|---|---|
| LPA1 antagonist | 2% |
| Hydroxyethylcellulose | 1% |
| Boric acid | 0.4% |
| Benzalkonium chloride | 0.01% |
| Sterile water for inhalation | q.s. |
| Disodium edetate | 0.01% |
| Phosphate buffer | to pH 7.0 |

LPA1 antagonist and sodium chloride are mixed together in water and the pH of the solution was adjusted to 7.4 by the addition of phosphate buffer. The vehicle was prepared by mixing together disodium edetate and hydroxyethylcellulose in water. Benzalkonium chloride was added to the mixture. A portion of this vehicle was added to the solution containing LPA1 antagonist and the mixture was packaged in sterile eyedrop dispensers.

### Example 3

### Ocular gel formulation

| Ingredient | Concentration (wt %) |
|---|---|
| LPA1 antagonist | 0.1% |
| mannitol | 3.6% |
| carbopol | 3.0% |
| thiomersal | 0.01% |
| Purified water | q.s. |
| Span 85 | 1% |
| Sodium acetate dihydrate | 0.1% |

LPA1 antagonist is suspended in water, Span 85 is added followed by addition of carbopol, sodium acetate and mannitol. Benzalkonium chloride is added, pH is adjusted to 5 with HCl/NaOH and the mixture is mixed well and packaged in sterile containers.

### Example 4

### Ocular Solution Formulation

| Ingredient | Concentration (wt %) |
|---|---|
| LPA1 antagonist | 2% |
| Citrate buffer | Adjust pH to 5.2 |
| Propylene glycol | 3% |
| Polyvinyl alcohol | 0.25% |
| Disodium edetate | 0.05% |
| Purified water | q.s. |

LPA1 antagonist and other ingredients are mixed well in water. The pH of the solution is adjusted to 5.2 with citrate buffer.

### Example 5

### Ocular Solution Formulation

| Ingredient | Concentration (wt %) |
|---|---|
| LPA1 antagonist | 2% |
| Citrate buffer | Adjust pH to 5.2 |
| Glycerin | 2% |
| Hydroxypropyl-β-cyclodextrin | 10% |
| Disodium edetate | 0.05% |
| Purified water | q.s. |

LPA1 antagonist and other ingredients are mixed well in water. The pH of the solution is adjusted to 5.2 with citrate buffer.

### Example 6

### Oral Solution

An oral solution is prepared at 20 mg/mL of LPA1 antagonist. In one embodiment, an oral pharmaceutical composition is prepared with the following ingredients:
20 mg/mL of LPA1 antagonist
aqueous 10 mM Na₂CO₃
20% propylene glycol

The manufacturing process for the oral solutions of LPA1 antagonist described above is as follows: weigh the required amount of sodium carbonate and transfer to the container. Add the required amount of water to make a 10 mM solution and mix until dissolved. Weigh the required amount of propylene glycol and add this to the solution and mix until homogenous. Weigh the required amount of LPA1 antagonist and slowly add to the solution. Mix until all LPA1 antagonist is dissolved (sonicate, warm, or stir if necessary).

### Example 7

### Capsule Formulation

In one embodiment, a capsule formulation of LPA1 antagonist for administration to humans is prepared with the following ingredients:

| | | Quantity per | Quantity per |
|---|---|---|---|
| | | Size 4 Capsule | Size 1 Capsule |
| Component | Function | mg | mg |
| Compound 1 or | Active | 10 to 500 mg | 100 to 1000 mg |
| Compound 2 | | | |
| Hypromellose, USP | Capsule Shell | 1 capsule | 1 capsule |

The process to prepare LPA1 antagonist in a capsule is as follows: Weigh the required amount of LPA1 antagonist, add into the appropriate size capsule, and close capsule.

### Example 8

### Tablet Formulation

Non-limiting examples of immediate release tables that include either LPA1 antagonist are presented below.

| | %, w/w |
|---|---|
| LPA1 antagonist | 33.3 |
| Mannitol | 17.7 |
| Prosolv HD 90 | 45.0 |
| Croscarmellose Sodium | 3.0 |
| Sodium Stearyl Fumarate | 1.0 |
| Total | 100 |

All excipients (except lubricant) are added to a V-shell blender. Add LPA1 antagonist to the V-shell blender. Mix in the V-shell blender for approximately 15 minutes. Add lubricant and blend for 2 minutes. Tablet cores are compressed with a suitable press. During compression, individual and average tablet weight, hardness, thickness, and friability are monitored.

### Example 9

### Establishment of a CHO Cell Line Stably Expressing Human LPA₁

A 1.1 kb cDNA encoding the human LPA₁ receptor was cloned from human lung. Human lung RNA (Clontech Laboratories, Inc. USA) was reverse transcribed using the RETROscript kit (Ambion, Inc.) and the full-length cDNA for human LPA₁ was obtained by PCR of the reverse transcription reaction. The nucleotide sequence of the cloned human LPA₁ was determined by sequencing and confirmed to be identical to the published human LPA₁ sequence (An et al. Biochem. Biophys. Res. Commun. 231:619 (1997). The cDNA was cloned into the pcDNA5/FRT expression plasmid and transfected in CHO cells using lipofectamine 2000 (Invitrogen Corp., USA). Clones stably expressing human LPA₁ were selected using hygromycin and identified as cells that show Ca-influx in response to LPA.

### Example 10

### Generation of Cells Transiently Expressing Human hLPA₂

An expression vector encoding the human LPA₂ cDNA was transiently transfected into B103 cells using Lipofectamine™ 2000 (Invitrogen) following the manufacturers instruction. On the day before the assay, 30,000-35,000 cells/well were seeded together with 0.2 μl lipofectamine 2000 and 0.2 μg human LPA2 expression vector in 96-well Poly-D-Lysine coated black-wall clear-bottom plates (BD BioCoat) in DMEM+10% FBS. Following an overnight culture, cells were washed once with PBS then cultured in serum-free media for 4 hours prior to start of the calcium flux assay.

### Example 11

### Establishment of a CHO Cell Line Stably Expressing Human LPA₃

A vector containing the human LPA₃ receptor cDNA was obtained from the Missouri S&T cDNA Resource Center (www.cdna.org). The full-length cDNA fragment for human LPA₃ was obtained by PCR from the vector. The nucleotide sequence of the cloned human LPA₃ was determined by sequencing and confirmed to be identical to the published human LPA₃ sequence (NCBI accession number NM_{—}012152). The cDNA was cloned into the pcDNA5/FRT expression plasmid and transfected in CHO cells using lipofectamine 2000 (Invitrogen Corp., USA). Clones stably expressing human LPA₃ were selected using hygromycin and identified as cells that show Ca-influx in response to LPA.

### Example 12

### LPA1 and LPA3 Calcium Flux Assays

Human LPA₁ or LPA₃ expressing CHO cells are seeded at 20,000-45,000 cells per well in a 96-well poly-D-lysine coated plate one or two days before the assay. Prior to the assay, the cells are washed once with PBS and then cultured in serum-free media for at least 6 hrs and up to 24 hrs. On the day of the assay, a calcium indicator dye (Calcium 5, Molecular Devices) in assay buffer (HBSS with Ca²⁺ and Mg²⁺ and containing 20 mM Hepes and 0.3% fatty-acid free human serum albumin) is added to each well and incubation continued for 1 hour at room temperature. 10 μl of test compounds in 2.5% DMSO are added to the cells and incubation continued at room temperature for 30 minutes. Cells are the stimulated by the addition of 10 nM LPA and intracellular Ca²⁺ measured using the Flexstation 3 (Molecular Devices). IC₅₀s are determined using Symyx Assay Explorer analysis of drug titration curves.

### Example 13

### LPA2 Calcium Flux Assay

LPA2 calcium flux is measured using at least one of two different assays. In one assay, BT-20 human breast cancer cells are seeded at 25,000-35,000 cells per well in 150 μl complete media on Poly-D-Lysine coated black-wall clear-bottom plates. Following an overnight culture, cells are washed once with PBS then serum starved for 4-6 hours prior to the assay. On the day of the assay, a calcium indicator dye (Calcium 5, Molecular Devices) in assay buffer (HBSS with Ca²⁺ and Mg²⁺ and containing 20 mM Hepes and 0.3% fatty-acid free human serum albumin) is added to each well and incubation continued for 15 minutes at 37° C. 25 μl of test compounds in 2.5% DMSO are added to the cells and incubation continued at 37° C. for 15-30 minutes. Cells are the stimulated by the addition of 100 nM LPA and intracellular Ca²⁺ measured using the Flexstation 3 (Molecular Devices). IC₅₀s are determined using Symyx Assay Explorer analysis of drug titration curves In the second assay, B103 cells transiently expressing huma LPA2 are serum starved for 4 hours. A calcium indicator dye (Calcium 4, Molecular Devices) in assay buffer (HBSS with Ca²⁺ and Mg²⁺ and containing 20 mM Hepes and 0.3% fatty-acid free human serum albumin) is then added to each well and incubation continued for 1 hour at 37° C. 10 μl of test compounds in 2.5% DMSO are added to the cells and incubation continued at room temperature for 30 minutes. Cells are the stimulated by the addition of 10 nM LPA and intracellular Ca²⁺ measured using the Flexstation 3 (Molecular Devices). IC₅₀s are determined using Symyx Assay Explorer analysis of drug titration curves.

### Example 14

### GTPγS Binding Assay

The ability of a compound to inhibit binding of GTP to LPA₁ is assessed via a membrane [³⁵S]-GTPγS binding assay. CHO cells stably expressing the recombinant human LPA₁ receptor are resuspended in 10 mM Hepes, 7.4 containing 1 mM DTT, lysed and centrifuged at 75,000×g to pellet the membranes. The membranes are resuspended in 10 mM Hepes, 7.4 containing 1 mM DTT and 10% glycerol. Membranes (˜25 μg per well) are incubated in 96-well plates with 0.1 nM [³⁵S]-GTPγS, 900 nM LPA, 5 μM GDP, and test compound in Assay Buffer (50 mM Hepes, pH 7.4, 100 mM NaCl, 10 mM MgCl₂, 50 μg/ml saponin and 0.2% fatty-acid free human serum albumin) for 30 minutes at 30° C. The reactions are terminated by rapid filtration through Whatman GF/B glass fibre filter plates. The filter plates are washed 3 times with 1 ml cold Wash Buffer (50 mM Hepes, 7.5, 100 mM NaCl and 10 mM MgCl₂) and dried. Scintillant is then added to the plates and the radioactivity retained on the filters is determined on a Packard TopCount (Perkin Elmer). Specific binding is determined as total radioactive binding minus non-specific binding in the absence of the ligand (900 nM LPA). IC₅₀s were determined using Graphpad prism analysis of drug titration curves.

Illustrative in vitro biological data is presented in the Table below.

| | Ca Flux IC₅₀ (μM) | | | | |
|---|---|---|---|---|---|
| | LPA₁ | LPA₂ | LPA₃ | LPA₄ | LPA₅ |
| Compound A | A | C | C | C | C |
| Compound B | A | C | C | C | C |
| Compound C | A | C | C | C | C |
| Compound D | A | B | C | C | C |
| Compound E | A | C | B | ND | C |
| Compound F | A | ND | C | C | ND |
| Compound G | A | C | C | C | C |
| Compound H | A | C | C | C | C |

A = less than 0.2 μM,
B = 0.2-1.0 μM, and
C = greater than 1 μM,
ND = assay not performed

### Example 15

### Choroidal Neovascularization Model

Animals: Sixty, 10-12 week old female C57BL6/J mice were purchased from Jackson Laboratories (Bar Harbor, Me.). All animal studies were performed under a protocol approved by the Institutional Animal Care and Use Committee at the University of Florida, and in accordance with the ARVO Statement for the Use of Animals in Ophthalmic and Vision Research.

Experimental agent: LPA1 receptor-selective antagonist.

Pharmacokinetic studies: Mice (n=10/timepoint) underwent laser-induced rupture of Bruch's membrane in on eye and the contralateral eye served as an uninjured control. On day 7 after laser rupture, mice received a single, oral (30 mg/kg) dose of Compound B, 2 and 24 hr prior to sacrifice to allow measurement of peak and trough drug concentrations. At each timepoint after oral dosing, mice were sacrificed and mouse plasma, neural retina and posterior cup (injured and uninjured eye) were isolated and frozen in dry ice for subsequent analysis of drug concentrations.

Drug levels in the injured eye were compared with that of the uninjured eye.

TABLE 1
PK study

| | | | | | Time of | Evaluations |
|---|---|---|---|---|---|---|
| Drug | | # of | Dose | Time of | Sacrifice | Post- |
| Treatment | Group | mice | (mg/kg) | dosing | post-laser | Sacrifice |
| Compound | A | 10 | 30 | −2 hr | day 7 | Drug levels |
| B | | | | | | in Plasma, |
| | | | | | | retina & |
| | | | | | | posterior cup |
| Compound | B | 10 | 30 | −24 hr | day 7 | Drug levels |
| B | | | | | | in Plasma, |
| | | | | | | retina & |
| | | | | | | posterior cup |

Efficacy studies: mice (n=100/treatment group/endpoint) underwent laser-induced rupture of Bruch's membrane in on eye and the contralateral eye served as an uninjured control. Dosing was initiated on the day of laser rupture. Mice were dosed orally by gavage, twice a day (BID) with Vehicle (water) or Compound B, or were dosed intravitreally (once a week) with Compound B (dissolved in sterile saline) for a period of 14 days after laser rupture.

TABLE II
Efficacy study

| | | | | Time | Time of | Evaluations |
|---|---|---|---|---|---|---|
| Drug | | # of | Dose | of | Sacrifice | Post- |
| Treatment | Group | mice | (mg/kg) | dosing | post-laser | Sacrifice |
| Vehicle | A | 20 | — | BID | Day 14 | CNV (n = 10) |
| (water) | | | | | | and fibrosis |
| | | | | | | (n = 10) |
| Compound | B | 20 | 30 | PO, | Day 14 | CNV (n = 10) |
| B | | | mg/kg | BID | | and fibrosis |
| | | | | | | (n = 10) |
| Compound | C | 20 | 3 | IVT, | Day 14 | CNV (n = 10) |
| B | | | μg/eye | Weekly | | and fibrosis |
| | | | | | | (n = 10) |

Quantification of CNV in the laser injury murine model: Choroidal neovascularization was evaluated as previously reported (Sengupta et al., The role of adult bone marrow-derived stem cells in choroidal neovascularization, Invest. Ophthalmol. Vis. Sci. 44 (11), pp. 4908-4913, 2003) Briefly, animals from each treatment cohort were euthanized by overdose of ketamine-xylazine mixture, and then underwent whole body perfusion via cardiac puncture with 6 ml 4% paraformaldehyde in PBS, pH 7.5. The eyes were enucleated, punctured with a 27 gauge needle 1 mm posterior to the limbus, and immersed in fixative for 1 h at room temperature, then washed two times by immersion in PBS buffer for 30 min. Eyes were then prepared for morphometry and volumetric measurement of CNV lesions as follows. The eyes were dissected to isolate the posterior segment consisting of the retinal pigment epithelium, the choriocapillaris and the sclera. This tissue was then permeabilized and reacted with rhodamine-conjugated Ricinus communis agglutinin I (Vector Laboratories, Burlingame, Calif.) to detect the vessels within the CNV lesion. The posterior cups were cut with 4-7 radial slices, and mounted flat on microscope slides with a drop of Vectashield anti-fade medium (Vector Laboratories) for digital image capture by epifluorescence Zeiss Axioplan 2 with RGB Spot high-resolution digital camera and laser scanning confocal microscopy (BioRad MRC 1024, BioRad Corporation, Temecula, Calif.).

Captured digital images were evaluated morphometrically using ImageJ software (Research Services Branch, National Institutes of Health, Bethesda, Md.). Each confocal z-series image capture of the red channel was analyzed as follows: (1) a calibration for the specific objective and microscope was applied to set the pixel-to-length ratio; (2) a threshold will be applied using the Otsu algorithm; (3) images were made binary; (4) a region-of-interest (ROI) was outlined to include the entire lesion area; (5) a particle analysis was performed to quantify the pixel area above the threshold level within the ROI. The sum of lesion area throughout the z-series was then multiplied by the z thickness (typically 4 μm) to obtain the lesion volume. The three lesion volumes for each animal were averaged and treated as an n of 1 for statistical analysis. Changes in lesion volume among treatment groups was determined by averaging the mean lesion volume for all animals in a treatment group, and reported as mean±standard error from the mean. Comparisons were tested for statistical significance by Student's t-test or one-way analysis of variance (ANOVA). Differences in lesion volume with a p value less than or equal to 0.05 were considered significant.

Collagen staining of CNV lesions: Selected mouse eyes from each treatment group were fixed by immersion in Trump's fixative overnight, followed by three changes into PBS, after which they were embedded in paraffin and the blocks sectioned. The entire globes were cut at 8 μm per section, collecting every tenth section onto coated microscope slides (SuperfrostPlus, Fisher Scientific, Orlando, Fla.). The sections were then stained using Masson's Trichrome (Dako Corp., Carpenteria, Calif.) and digital images of each entire section were captured. The calibrated images were examined by masked observers who selected the sections that showed the largest lesion diameter. To determine collagen deposition (scar formation) within the CNV lesion, the cross-sectional area (μm²) of Trichrome staining within each sequential section was measured using ImageJ and the values from all sections were averaged to determine representative collagen deposition within the entire CNV lesion, and analyzed statistically as described above.

Compound B decreased CNV lesion volume by 11% (non-significant) and 39% (significant, p<0.05), respectively, in treatment groups B and C.

### Example 16

### Mouse db/db Model of Diabetic Retinopathy

A mouse model of diabetic retinopathy was used to evaluate the effect of compound 3 on ocular complications secondary to diabetes. Male non-insulin dependent diabetic mellitus (NIDDM) mice (BKS Cg-Lepr db/Lepr db) weighing 50±10 g were used at an age of 15 weeks. Animals received either vehicle (distilled water) or Compound 3 (3 and 30 mg/kg) by oral gavage twice daily for eight consecutive weeks. All animals were allowed free access to normal laboratory chow and water throughout the experiment. Serum glucose and insulin levels were determined by enzymatic method (Mutaratase-GOD) and ELISA (mouse insulin assay kit) from orbital sinus blood samples obtained on weeks 1 and 8. At study completion animals were sacrificed and the right eye preserved for histolopathology by immersing in 10% formalin and the left eye snap frozen for biochemical analysis. Type IV collagen was determined using a competitive immunoassay using rabbit anti-mouse type IV collagen antibody after thawing the fresh frozen eye and homogenizing in 100 μl of buffer containing 50 mM Tris-HCL, pH 7.5, 150 mM NaCl, 0.05% NP-40, 0.1% proteinase inhibitor and 0.5 mM phenylmethylsulfonyl fluoride. The formalin fixed eye was evaluated for retinal thickness using quantitative morphometric analysis of the thickness of the retinal capillary basement basement membrane. All data are reported as means±standard error of the mean and analyzed by t-test or ANOVA where appropriate.

### Example 17

### Clinical Trial

Clinical Trial Evaluating Effect of Topical Administration of an LPA receptor antagonist Compound to the Eye in Reducing Signs and Symptoms of Corneal Scarring After Photorefractive Keratectomy for High Myopia: 15×30 Seconds.

A single-center, double-blind, randomized, two way cross-over, placebo-controlled study to evaluate the safety and efficacy of topical administration of LPA receptor antagonist compound to the eye of individuals corneal scarring following Photorefractive Keratectomy for High Myopia: 15×30 Seconds.

Each subject will receive the active treatment (e.g. a topical formulation of a LPA antagonist compound administered to the eye) or placebo.

Study Protocol:

30 subjects aged 18-65 years of either gender are to participate in the study. Patients in both treatment groups will be treated with an ophthalmic solution formulation of example 5, two drops instilled in the eye three times daily for a period of two months.

Inclusion Criteria:

Myopia or compound myopic astigmatism; Stable refractive error; Pupil size 6 mm in room light, No associated eye disease; photorefractive keratectomy within 30 days prior to start of study.

Exclusion Criteria:

Diabetes, Autoimmune diseases, Topographic abnormalities.

The primary endpoint of the study is the improvement in vision in the treated versus placebo groups, incidence of corneal haze and/or corneal scarring, incidence of dry eye, glare, halos or starburst aberrations.

Secondary endpoints include individual ocular signs and symptoms as assessed by the patient and the physician, the overall assessment of the patient, the overall assessment of the physician, the overall assessment of visual acuity, safety and tolerability, decrease in the healing period, and reduction in pain associated with the surgery.

The examples and embodiments described herein are for illustrative purposes only and various modifications or changes suggested to persons skilled in the art are to be included within the spirit and purview of this application and scope of the appended claims.

What is claimed is:

1. An ophthalmic formulation comprising an LPA1 receptor antagonist and at least one suitable pharmaceutically acceptable excipient, wherein the formulation is in a form suitable for administration to the eye of a mammal.

2. The ophthalmic formulation of claim 1, wherein the LPA1 receptor antagonist is in an amount effective for the treatment of an ophthalmic disease or condition in a mammal, and the ophthalmic formulation is in the form of a solution, suspension, emulsion, ointment, cream, lotion, gel, colloidal dispersion, or spray.

3. (canceled)

4. The ophthalmic formulation of claim 1, wherein the ophthalmic formulation is in the form of a solution that is administered to the mammal in the form of an eye drop or eye ointment.

5. (canceled)

6. The ophthalmic formulation of claim 1, wherein the LPA1 receptor antagonist is a compound having the structure of Formula (I) wherein, 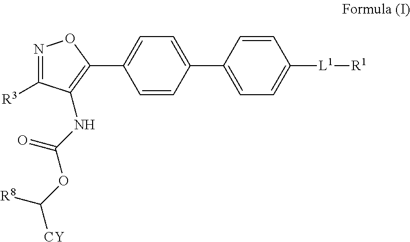
R¹ is —CO₂H, —CO₂R^{D}, tetrazolyl, 5-oxo-2,5-dihydro-[1,2,4]oxadiazol-3-yl, —C(═O)NHSO₂CH₃, or —C(═O)NHSO₂CH₂CH₃; R^{D} is —CH₃, —CH₂CH₃, —CH₂CH₂CH₃, —CH(CH₃)₂, —CH₂CH₂CH₂CH₃, or —C(CH₃)₃;
L¹is C₁-C₄alkylene or C₃-C₆cycloalkylene;
R³ is H, —CH₃, —CH₂CH₃, or —CF₃;
R⁸ is H or —CH₃;
CY is C₁-C₆alkyl, substituted or unsubstituted C₃-C₆cycloalkyl, or substituted or unsubstituted phenyl; wherein if CY is substituted then CY is substituted with 1 or 2 R^{C};
each R^{C} is independently F, Cl, Br, I, —OH, —CN, C₁-C₄alkyl, C₁-C₄fluoroalkyl, C₁-C₄fluoroalkoxy, or C₁-C₄alkoxy;
or a pharmaceutically acceptable salt thereof.

7. The ophthalmic formulation of claim 6, wherein the compound of Formula (I) has the following structure:

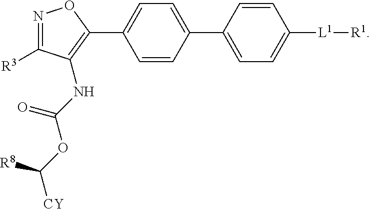

8. The ophthalmic formulation of claim 6, wherein the compound of Formula (I) has the structure of Formula (II): 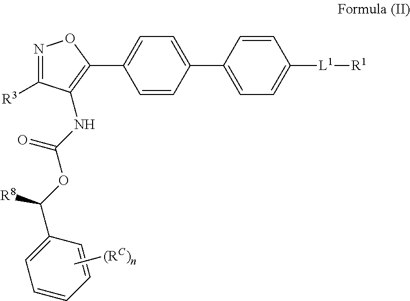
wherein,
n is 0, 1, or 2.

9. (canceled)

10. The ophthalmic formulation of claim 6, wherein the LPA1 receptor antagonist is:
(R)-2-(4′-(3-methyl-4-((1-phenylethoxy)carbonylamino)isoxazol-5-yl)biphenyl-4-yl)acetic acid (Compound A):
(R)-1-(4′-(3-methyl-4-((1-phenylethoxy)carbonylamino)isoxazol-5-yl)biphenyl-4-yl)cyclopropanecarboxylic acid (Compound B):
(R)-2-(4′-(4-((1-(2-chlorophenyl)ethoxy)carbonylamino)-3-methylisoxazol-5-yl)biphenyl-4-yl)acetic acid (Compound C):
{5-[4′-(1-Methanesulfonylaminocarbonyl-cyclopropyl)-biphenyl-4-yl]-3-methyl-isoxazol-4-yl}-carbamic acid (R)-1-phenyl-ethyl ester (Compound D):
1-(4′-{4-[(R)-1-(2-Chloro-phenyl)-ethoxycarbonylamino]-3-methyl-isoxazol-5-yl}-biphenyl-4-yl)-cyclopropanecarboxylic acid (Compound E):
1-{4′-[4-((R)-1-Phenyl-ethoxycarbonylamino)-isoxazol-5-yl]-biphenyl-4-yl}-cyclopropanecarboxylic acid (Compound F):
(3-Methyl-5-{4′-[1-(5-oxo-2,5-dihydro-[1,2,4]oxadiazol-3-yl)-cyclopropyl]-biphenyl-4-yl}-isoxazol-4-yl)-carbamic acid (R)-1-phenyl-ethyl ester (Compound G):
(3-Methyl-5-{4′-[1-(1H-tetrazol-5-yl)-cyclopropyl]-biphenyl-4-yl}-isoxazol-4-yl)-carbamic acid (R)-1-phenyl-ethyl ester (Compound H);
or a pharmaceutically acceptable salt thereof.

11. The ophthalmic formulation of claim 1 wherein the LPA1 receptor antagonist has structure of Formula (III): 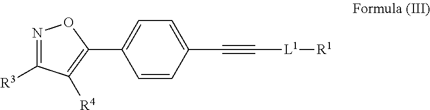
wherein
R¹ is —CO₂H, —CO₂R^{D}, tetrazolyl, 5-oxo-2,5-dihydro-[1,2,4]oxadiazol-3-yl, —C(═O)NHSO₂CH₃, or —C(═O)NHSO₂CH₂CH₃; R^{D} is —CH₃, —CH₂CH₃, —CH₂CH₂CH₃, —CH(CH₃)₂, —CH₂CH₂CH₂CH₃, or —C(CH₃)₃;
L¹ is absent, or a C₁-C₆alkylene;
R³ is H, —CH₃, —CH₂CH₃, or —CF₃;
R⁴ is —NHC(═O)OCH(R⁸)—CY;
R⁸ is H, or —CH₃;
CY is substituted or unsubstituted phenyl; wherein if CY is substituted then CY is substituted with 1 or 2 R^{C}; each R^{C} is independently F, Cl, Br, I, —OH, —CN, C₁-C₄alkyl, C₁-C₄fluoroalkyl, C₁-C₄fluoroalkoxy, or C₁-C₄alkoxy;
or a pharmaceutically acceptable salt thereof.

12. (canceled)

13. The ophthalmic formulation of claim 11, wherein the LPA1 receptor antagonist is 6-(4-{4-[1-(2-Chloro-phenyl)-ethoxycarbonylamino]-3-methyl-isoxazol-5-yl}-phenyl)-hex-5-ynoic acid, or 7-(4-{4-[1-(2-chloro-phenyl)-ethoxycarbonylamino]-3-methyl-isoxazol-5-yl}-phenyl)-hept-6-ynoic acid, or a pharmaceutically acceptable salt thereof.

14. The ophthalmic formulation of claim 1, wherein the LPA1 receptor antagonist has the structure of Formula (VI): 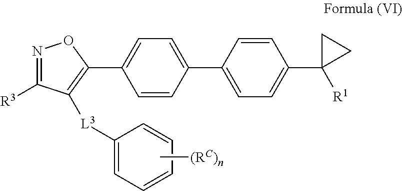
R¹ is —CO₂R^{D}, —C(═O)NHSO₂R^{E}, —C(═O)N(R^{D})₂, or tetrazolyl;
R^{D} is H or C₁-C₆alkyl;
R^{E} is C₁-C₆alkyl, C₃-C₆cycloalkyl, or substituted or unsubstituted phenyl;
L³ is a substituted or unsubstituted C₃-C₆alkylene, a substituted or unsubstituted C₃-C₆-fluoroalkylene, or a substituted or unsubstituted C₃-C₆heteroalkylene, where if L³ is substituted then L³ is substituted with 1, 2 or 3 R¹³; each R¹³ is independently F, C₁-C₄alkyl, C₁-C₄fluoroalkyl, or —OH;
each R^{C} is independently halogen, —CN, —NO₂, —OH, C₁-C₄alkyl, C₁-C₄fluoroalkyl, C₁-C₄fluoroalkoxy, C₁-C₄alkoxy, or C₁-C₄heteroalkyl;
R³ is H or C₁-C₄alkyl;
n is 0, 1, or 2;
or a pharmaceutically acceptable salt thereof.

15. The ophthalmic formulation of claim 14, wherein:
R¹ is —CO₂R^{D}, or —C(═O)NHSO₂R^{E};
R^{D} is H or C₁-C₄alkyl;
R^{E} is C₁-C₄alkyl;
L³ is a substituted or unsubstituted C₃-C₄alkylene, a substituted or unsubstituted C₃-C₄fluoroalkylene, or a substituted or unsubstituted C₃-C₆heteroalkylene; where if L³ is substituted then L³ is substituted with 1, 2 or 3 R¹³; each R¹³ is independently selected from F, —CH₃, —CH₂CH₃, —CF₃, and —OH;
R³ is —H, —CH₃ or —CH₂CH₃.

16. The ophthalmic formulation of claim 1 wherein the LPA1 receptor antagonist has the structure of Formula (VII): 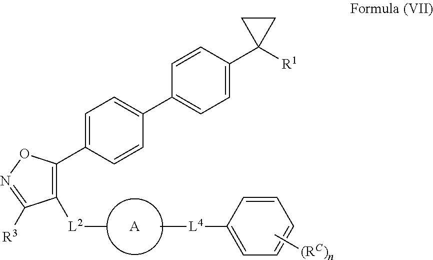
wherein,
R¹ is —CO₂R^{D}, —C(═O)NHSO₂R^{E}, —C(═O)N(R^{D})₂, —CN, or tetrazolyl;
R^{D} is H or C₁-C₆ alkyl;
R^{E} is C₁-C₆ alkyl or a substituted or unsubstituted phenyl;
L² is absent, —C(═O)—, —N(R^{D})—, substituted or unsubstituted C₁-C₄ alkylene, or substituted or unsubstituted C₁-C₄ heteroalkylene, where if L² is substituted, then L² is substituted with R¹², where R¹² is F, C₁-C₄alkyl, —OH, or —OR^{D};
ring A is a substituted or unsubstituted phenyl, or a substituted or unsubstituted monocyclic C₁-C₅heteroarylene, where if ring A is substituted, then ring A is substituted with 1 or 2 R¹⁴, each R¹⁴ is independently selected from halogen, —CN, —OH, C₁-C₄alkyl, C₁-C₄fluoroalkyl, C₁-C₄fluoroalkoxy, C₁-C₄alkoxy, and C₁-C₄heteroalkyl;
L⁴ is absent, or a substituted or unsubstituted C₁-C₄ alkylene, where if L⁴ is substituted then L⁴ is substituted with R¹³, where R¹³ is F, C₁-C₄alkyl, —OH, or —OR^{D};
R³ is H or C₁-C₄ alkyl;
each R^{C} is independently selected from halogen, —CN, —OH, C₁-C₄alkyl, C₁-C₄fluoroalkyl, C₁-C₄fluoroalkoxy, C₁-C₄alkoxy, and C₁-C₄heteroalkyl;
n is 0, 1 or 2;
or a pharmaceutically acceptable salt thereof.

17. The ophthalmic formulation of claim 16, wherein:
R¹ is —CO₂R^{D}, or —C(═O)NHSO₂R^{E};
R^{D} is H or C₁-C₄ alkyl;
R^{E} is C₁-C₄ alkyl;
L² is —CH₂—, —CH(CH₃)—, or —CH(OH)—;
ring A is a substituted or unsubstituted 5-membered monocyclic C₁-C₄heteroarylene containing 1-4 N atoms, 0 or 1 O atoms and 0 or 1 S atoms, where if ring A is substituted, then ring A is substituted with R¹⁴;
L⁴ is —CH₂— or —CH(CH₃)—;
p is 0 or 1.

18. The ophthalmic formulation of claim 16, wherein:
R¹ is —CO₂R^{D}, or —C(═O)NHSO₂R^{E};
R^{D} is H or C₁-C₄alkyl;
R^{E} is C₁-C₄alkyl;
L² is —NH—, —CH₂—, —CH(CH₃)—, —CH(OH)—, —NHCH₂— or —NHCH(CH₃)—;
ring A is a substituted or unsubstituted 6-membered monocyclic C₃-C₅heteroarylene containing 1-3 N atoms, where if ring A is substituted, then ring A is substituted with R¹⁴;
L⁴ is absent, —CH₂—, or —CH(CH₃)—;
p is 0 or 1.

19. (canceled)

20. (canceled)

21. (canceled)

22. (canceled)

23. (canceled)

24. A method of treating an ocular disease or condition in a mammal, comprising administering to the mammal in need thereof a therapeutically-effective amount of an LPA1 receptor antagonist.

25. (canceled)

26. (canceled)

27. The method of claim 24, wherein the ocular disease or condition is ocular hypertension, primary open-angle glaucoma, episcleral fibrosis leading to trabeculectormy (bleb) failure after glaucoma filtration surgery, dry eyes, Sjogren syndrome, inflammation following ocular surgery, keratoconjuctivitis, pterygia, non-specific orbital inflammation, cataracts, post-surgical corneal scarring, corneal scarring, scarring associated with ocular cicatricial pemphigoid, glaucoma filtration surgery, thyroid eye disease, anterior uveitis, or fibrosis associated with keratoprosthesis procedure.

28. The method of claim 24, wherein the LPA1 receptor antagonist is a compound having the structure of Formula (I) 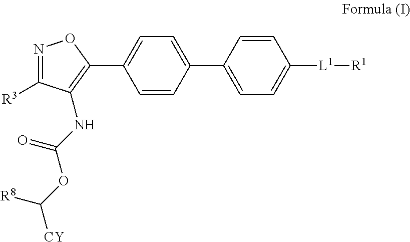
wherein
R¹ is —CO₂H, —CO₂R^{D}, tetrazolyl, 5-oxo-2,5-dihydro-[1,2,4]oxadiazol-3-yl, —C(═O)NHSO₂CH₃, or —C(═O)NHSO₂CH₂CH₃; R^{D} is —CH₃, —CH₂CH₃, —CH₂CH₂CH₃, —CH(CH₃)₂, —CH₂CH₂CH₂CH₃, or —C(CH₃)₃;
L¹ is C₁-C₄alkylene or C₃-C₆cycloalkylene;
R³ is H, —CH₃, —CH₂CH₃, or —CF₃;
R⁸ is H or —CH₃;
CY is C₁-C₆alkyl, substituted or unsubstituted C₃-C₆cycloalkyl, or substituted or unsubstituted phenyl; wherein if CY is substituted then CY is substituted with 1 or 2 R^{C};
each R^{C} is independently F, Cl, Br, I, —OH, —CN, C₁-C₄alkyl, C₁-C₄fluoroalkyl, C₁-C₄fluoroalkoxy, or C₁-C₄alkoxy;
or a pharmaceutically acceptable salt thereof.

29. (canceled)

30. (canceled)

31. (canceled)

32. (canceled)

33. The method of claim 24 wherein the LPA1 receptor antagonist has structure of Formula (III): 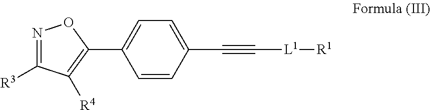
wherein
R¹ is —CO₂H, —CO₂R^{D}, tetrazolyl, 5-oxo-2,5-dihydro-[1,2,4]oxadiazol-3-yl, —C(═O)NHSO₂CH₃, or —C(═O)NHSO₂CH₂CH₃; R^{D} is —CH₃, —CH₂CH₃, —CH₂CH₂CH₃, —CH(CH₃)₂, —CH₂CH₂CH₂CH₃, or —C(CH₃)₃;
L¹ is absent, or a C₁-C₆alkylene;
R³ is H, —CH₃, —CH₂CH₃, or —CF₃;
R⁴ is —NHC(═O)OCH(R⁸)—CY;
R⁸ is H, or —CH₃;
CY is substituted or unsubstituted phenyl; wherein if CY is substituted then CY is substituted with 1 or 2 R^{C}; each R^{C} is independently F, Cl, Br, I, —OH, —CN, C₁-C₄alkyl, C₁-C₄fluoroalkyl, C₁-C₄fluoroalkoxy, or C₁-C₄alkoxy;
or a pharmaceutically acceptable salt thereof.

34. (canceled)

35. The method of claim 33, wherein the LPA1 receptor antagonist is 6-(4-{4-[1-(2-Chloro-phenyl)-ethoxycarbonylamino]-3-methyl-isoxazol-5-yl}-phenyl)-hex-5-ynoic acid, or 7-(4-{4-[1-(2-chloro-phenyl)-ethoxycarbonylamino]-3-methyl-isoxazol-5-yl}-phenyl)-hept-6-ynoic acid, or a pharmaceutically acceptable salt thereof.

36. The method of claim 24 wherein the LPA1 receptor antagonist has the structure of Formula (VI): 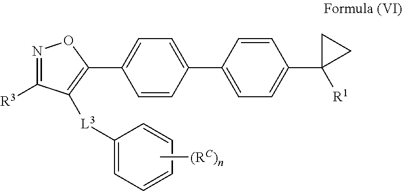
R¹ is —CO₂R^{D}, —C(═O)NHSO₂R^{E}, —C(═O)N(R^{D})₂, or tetrazolyl;
R^{D} is H or C₁-C₆alkyl;
R^{E} is C₁-C₆alkyl, C₃-C₆cycloalkyl, or substituted or unsubstituted phenyl;
L³ is a substituted or unsubstituted C₃-C₆alkylene, a substituted or unsubstituted C₃-C₆fluoroalkylene, or a substituted or unsubstituted C₃-C₆heteroalkylene, where if L³ is substituted then L³ is substituted with 1, 2 or 3 R¹³; each R¹³ is independently F, C₁-C₄alkyl, C₁-C₄fluoroalkyl, or —OH;
each R^{C} is independently halogen, —CN, —NO₂, —OH, C₁-C₄alkyl, C₁-C₄fluoroalkyl, C₁-C₄fluoroalkoxy, C₁-C₄alkoxy, or C₁-C₄heteroalkyl;
R³ is H or C₁-C₄alkyl;
n is 0, 1, or 2;
or a pharmaceutically acceptable salt thereof.

37-46. (canceled)
